(19) United States
(12) Patent Application Publication
(10) Pub. No.: US 2019/0262496 A1
(43) Pub. Date: Aug. 29, 2019

(54) Compositions and Methods for Collagen-Based Sutures and Antibacterial Coatings

(71) Applicant: UNIVERSITY HOSPITALS CLEVELAND MEDICAL CENTER, Cleveland, OH (US)

(72) Inventors: Chad ZENDER, Cleveland, OH (US); Olivia CHENG, Solon, OH (US); Ozan AKKUS, Cleveland, OH (US); Eric BABAJANIAN, Cleveland, OH (US)

(21) Appl. No.: 16/317,643

(22) PCT Filed: Jul. 14, 2017
(86) PCT No.: PCT/US2017/042146

§ 371 Date: Jan. 14, 2019

Related U.S. Application Data
(60) Provisional application No. 62/362,234, filed on Jul. 14, 2016.

(51) Int. Cl. A61L 17/08; A61L 27/34; A61L 27/54; A61L 31/04; A61L 31/16; A61L 17/00

(52) U.S. Cl. CPC A61L 17/08; A61L 27/34; A61L 27/54; A61L 31/044; A61L 2300/414; A61L 17/005; A61L 2300/236; A61L 2300/406; A61L 31/16

(57) ABSTRACT

This invention is related to the field of medical device implants. In particular, the invention is related to sutures and antibacterial coatings that comprise collagen. In particular, some of the collagen-based coatings prevent and control of bacterial infections that result from the use of medical device implants (e.g., head and neck implants). One method of preventing post-medical device implant bacterial infections comprises using a medical device coated with a biologic antimicrobial coating. For example, one such coating comprises a covalently crosslinked heparin-collagen sheet.

### FIELD OF THE INVENTION

This invention is related to the field of medical device implants. In particular, the invention is related to sutures and antibacterial coatings that comprise collagen. In particular, some of the collagen-based coatings prevent and control of bacterial infections that result from the use of medical device implants (e.g., head and neck implants). One method of preventing post-medical device implant bacterial infections comprises using a medical device coated with a biologic antimicrobial coating. For example, one such coating comprises a covalently crosslinked heparin-collagen sheet.

### BACKGROUND

Infection is one of the most common and devastating complications associated with the implantation of medical devices. Implants provide a surface on which many bacterial species are able to adhere and produce a biofilm matrix. Given the tremendous human cost and financial burden of these infections, the projected increase in the use of implantable devices, and the growing problem of bacterial resistance, the development of strategies to prevent device-related infections is more pressing than ever.

Recently, a variety of methods have been developed as part of a growing interest in localized infection prophylaxis. There are two major categories: the incorporation of antibacterial agents into the surface materials of devices and the introduction of local antibiotic delivery systems. Zilberman et al., (2008). Though some of these methods have been shown to have efficacy, they are limited by poorly controlled antibiotic release, limited chemical stability, local tissue toxicity, poor osteointegration, and inhibition of osteoblastic activity. Simchi et al. (2011). There has been a lack of evidence for the efficacy of systemic antibiotic prophylaxis While a variety of methods for localized infection prophylaxis have shown more promise, these have been limited in a number of ways.

To address these shortcomings, what is needed in the art is a shape-adaptable, biocompatible and biodegradable antimicrobial coating for medical devices

### SUMMARY OF THE INVENTION

This invention is related to the field of medical device implants. In particular, the invention is related to sutures and antibacterial coatings that comprise collagen. In particular, some of the collagen-based coatings prevent and control of bacterial infections that result from the use of medical device implants (e.g., head and neck implants). One method of preventing post-medical device implant bacterial infections comprises using a medical device coated with a biologic antimicrobial coating. For example, one such coating comprises a covalently crosslinked heparin-collagen sheet.

In one embodiment, the present invention contemplates an electrochemically aligned and compacted (ELAC) collagen suture comprising at least one growth factor and heparin. In one embodiment, the suture further comprises a crosslinker. In one embodiment, the crosslinker is genipin. In one embodiment, the at least one growth factor is conjugated to the ELAC suture. In one embodiment, the at least one growth factor saturates the ELAC suture. In one embodiment, the heparin is attached to the at least one growth factor. In one embodiment, the heparin attachment to the at least one growth factor is an affinity bonding attachment. In one embodiment, the at least one growth factor is platelet derived growth factor. In one embodiment, the at least one growth factor is selected from the group consisting of basic fibroblast growth factor, transforming growth factor-β1, vascular endothelial growth factor and insulin-like growth factor 1. In one embodiment, the ELAC suture further comprises a plurality of aligned threads. In one embodiment, the conjugation of the at least one growth factor to the ELAC suture is reversible. In one embodiment, the attachment of the at least one growth factor to the heparin is reversible. In one embodiment, the ELAC suture is a load-bearing ELAC suture. In one embodiment, the ELAC suture further comprises a heparinized collagen matrix. In one embodiment, the ELAC suture further comprises a plurality of genipin crosslinks. In one embodiment, the heparin is attached to the ELAC suture with peracetic acid in combination with 3-dimethylaminopropyl)-3-ethylcarbodiimide-N-hydroxysuccinimide.

In one embodiment, the present invention contemplates a method, comprising: a) providing; i) a patient exhibiting a wound, said wound comprising biological cells; and ii) an electrochemically aligned and compacted (ELAC) collagen suture comprising a plurality of at least one growth factor and heparin; b) suturing the wound with the suture to create a sutured wound; and c) healing the sutured wound faster than a conventional suture. In one embodiment, the suture further comprises a crosslinker. In one embodiment, the crosslinker is genipin. In one embodiment, the wound is a lacerated tendon. In one embodiment, the conventional suture is a nylon suture. In one embodiment, the conventional suture is a silk suture. In one embodiment, the method further comprising releasing the plurality of at least one growth factor from the ELAC suture. In one embodiment, the at least one growth factor release is approximately fifteen (15) days. In one embodiment, the at least one growth factor release induces proliferation of the biological cells. In one embodiment, the ELAC suture induces collagen production by the biological cells. In one embodiment, the ELAC suture is a load-bearing suture. In one embodiment, the ELAC suture upregulates Collagen I, SCX, COMP, and TNMD gene expression in the biological cells. In one embodiment, the biological cells are selected from the group consisting of tenocytes, tenoblasts and mesenchymal stem cells. In one embodiment, the at least one growth factor is conjugated to the ELAC suture. In one embodiment, the at least one growth factor saturates the ELAC suture. In one embodiment, the heparin is attached to the at least one growth factor. In one embodiment, the heparin attachment to the at least one growth factor is an affinity bonding attachment. In one embodiment, the at least one growth factor is platelet derived growth factor. In one embodiment, the at least one growth factor is selected from the group consisting of basic fibroblast growth factor, transforming growth factor-β1, vascular endothelial growth factor and insulin-like growth factor 1. In one embodiment, the ELAC suture further comprises a plurality of aligned threads. In one embodiment, the conjugation of the at least one growth factor to the ELAC suture is reversible. In one embodiment, the attachment of the at least one growth factor to the heparin is reversible. In one embodiment, the ELAC suture is a load-bearing ELAC suture. In one embodiment, the ELAC suture further comprises a heparinized collagen matrix. In one embodiment, the ELAC suture further comprises a plurality of genipin crosslinks. In one embodiment, the heparin is attached to the ELAC suture with peracetic acid in combination with 3-dimethylaminopropyl)-3-ethylcarbodiimide-N-hydroxysuccinimide.

In one embodiment, the present invention contemplates a biologic antibacterial coating composition comprising a heparin crosslinked collagen sheet. In one embodiment, the sheet further comprises gentamicin attached to the heparin. In one embodiment, the gentamicin-heparin attachment is an electrostatic association. In one embodiment, the sheet further comprises a drug or compound. In one embodiment, the drug or compound is an antibiotic. In one embodiment, the antibiotic includes, but is not limited to, a compound in one of the following classes, aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof.

In one embodiment, the present invention contemplates a medical device encased by a heparin crosslinked collagen sheet coating. In one embodiment, the coating further comprises gentamicin attached to said heparin. In one embodiment, the gentamicin-heparin attachment is an electrostatic association. In one embodiment, the coating further comprises a drug or compound. In one embodiment, the drug or compound is an antibiotic. In one embodiment, the antibiotic includes, but is not limited to, a compound in one of the following classes, aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof. In one embodiment, the medical device comprises a head implant. In one embodiment, the medical device comprises a neck implant. In one embodiment, the medical device is a titanium mandibular plate.

In one embodiment, the present invention contemplates a method, comprising: a) providing; i) a patient exhibiting a medical condition and a plurality of bacteria; ii) a medical device encased by a heparin crosslinked collagen sheet coating, said device being configured to treat said medical condition; and b) contacting said patient with said medical device under conditions such that said plurality of bacteria are subjected to a bacteriostatic effect. In one embodiment, said contacting further comprises that said plurality of bacteria are subjected to a bacteriocidal effect. In one embodiment, the coating further comprises gentamicin attached to said heparin. In one embodiment, the gentamicin-heparin attachment is an electrostatic association. In one embodiment, the coating further comprises a drug or compound. In one embodiment, the drug or compound is an antibiotic. In one embodiment, the antibiotic includes, but is not limited to, a compound in one of the following classes, aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof. In one embodiment, the medical device comprises a head implant. In one embodiment, the medical device comprises a neck implant. In one embodiment, the medical device is a titanium mandibular plate.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but also plural entities and also includes the general class of which a specific example may be used for illustration. The terminology herein is used to describe specific embodiments of the invention, but their usage does not delimit the invention, except as outlined in the claims.

The term “about” as used herein, in the context of any of any assay measurements refers to +/−5% of a given measurement.

The term “substitute for” as used herein, refers to the switching the administration of a first compound or drug to a subject for a second compound or drug to the subject.

The term “collagen sheet” as used herein, refers to any collagen composition made by an electrochemical processing of collagen-rich solutions which generate density backed and mechanically robust collagen layers. For example, a collagen sheet with geometrically complex patterns may form by 2D compaction of biomolecules, comprising a first electrode disposed a distance from a second electrode, and a patterned form positioned between the electrodes, wherein the patterned form is unaltered by a current applied to the electrodes. Process variables to create sheets (e.g., collagen sheets) include, but are not limited to, electric current density, electrode separation and density of the molecules, nanoparticles and microparticles with ampholytic nature in solution, which result in biophysical principles underlying an electrocompaction process.

The term “biologic antibacterial coating” as used herein, refers to any composition containing a material derived from a biological material (e.g., for example, collagen) that is combined with other compounds (e.g., for example, heparin) that confer antibacterial activity.

The term “encase” or “encased” as used herein, refers to completely surrounding an object with a material. The material may temporarily or permanently adhere, or attach, to the object. For example, a collagen sheet may completely surround and thereby encase a medical device implant.

The term “antibacterial activity” as used herein, refers to any effect of a drug or compound on a bacteria that either slows or stops growth (e.g., bacteriostatic effect) or actually kills the bacteria (bacteriocidal effect).

The term “suspected of having”, as used herein, refers a medical condition or set of medical conditions (e.g., preliminary symptoms) exhibited by a patient that is insufficent to provide a differential diagnosis. Nonetheless, the exhibited condition(s) would justify further testing (e.g., autoantibody testing) to obtain further information on which to base a diagnosis.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The term “effective amount” as used herein, refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds that exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The term “symptom”, as used herein, refers to any subjective or objective evidence of disease or physical disturbance observed by the patient. For example, subjective evidence is usually based upon patient self-reporting and may include, but is not limited to, pain, headache, visual disturbances, nausea and/or vomiting. Alternatively, objective evidence is usually a result of medical testing including, but not limited to, body temperature, complete blood count, lipid panels, thyroid panels, blood pressure, heart rate, electrocardiogram, tissue and/or body imaging scans.

The term “disease” or “medical condition”, as used herein, refers to any impairment of the normal state of the living animal or plant body or one of its parts that interrupts or modifies the performance of the vital functions. Typically manifested by distinguishing signs and symptoms, it is usually a response to: i) environmental factors (as malnutrition, industrial hazards, or climate); ii) specific infective agents (as worms, bacteria, or viruses); iii) inherent defects of the organism (as genetic anomalies); and/or iv) combinations of these factors.

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

The term “injury” as used herein, denotes a bodily disruption of the normal integrity of tissue structures. In one sense, the term is intended to encompass surgery. In another sense, the term is intended to encompass irritation, inflammation, infection, and the development of fibrosis. In another sense, the term is intended to encompass wounds including, but not limited to, contused wounds, incised wounds, lacerated wounds, non-penetrating wounds (i.e., wounds in which there is no disruption of the skin but there is injury to underlying structures), open wounds, penetrating wound, perforating wounds, puncture wounds, septic wounds, subcutaneous wounds, burn injuries etc.

The term “attached” as used herein, refers to any interaction between a medium (or carrier) and a drug. Attachment may be reversible or irreversible. Such attachment includes, but is not limited to, covalent bonding, ionic bonding, Van der Waals forces or friction, and the like. A drug is attached to a medium (or carrier) if it is impregnated, incorporated, coated, in suspension with, in solution with, mixed with, etc.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “pharmaceutically” or “pharmacologically acceptable”, as used herein, refer to molecular entities and compositions that do not produce adverse, allergic, or other untoward reactions when administered to an animal or a human.

The term, “pharmaceutically acceptable carrier”, as used herein, includes any and all solvents, or a dispersion medium including, but not limited to, water, ethanol, polyol (for example, glycerol, propylene glycol, and liquid polyethylene glycol, and the like), suitable mixtures thereof, and vegetable oils, coatings, isotonic and absorption delaying agents, liposome, commercially available cleansers, and the like. Supplementary bioactive ingredients also can be incorporated into such carriers.

The term “biocompatible”, as used herein, refers to any material does not elicit a substantial detrimental response in the host. There is always concern, when a foreign object is introduced into a living body, that the object will induce an immune reaction, such as an inflammatory response that will have negative effects on the host. In the context of this invention, biocompatiblity is evaluated according to the application for which it was designed: for example; a bandage is regarded a biocompable with the skin, whereas an implanted medical device is regarded as biocompatible with the internal tissues of the body. Preferably, biocompatible materials include, but are not limited to, biodegradable and biostable materials.

The term “biodegradable” as used herein, refers to any material that can be acted upon biochemically by living cells or organisms, or processes thereof, including water, and broken down into lower molecular weight products such that the molecular structure has been altered.

The term “bioerodible” as used herein, refers to any material that is mechanically worn away from a surface to which it is attached without generating any long term inflammatory effects such that the molecular structure has not been altered. In one sense, bioerosin represents the final stages of “biodegradation” wherein stable low molecular weight products undergo a final dissolution.

The term “bioresorbable” as used herein, refers to any material that is assimilated into or across bodily tissues. The bioresorption process may utilize both biodegradation and/or bioerosin.

The term “biostable” as used herein, refers to any material that remains within a physiological environment for an intended duration resulting in a medically beneficial effect. The term “medical device”, as used herein, refers broadly to any apparatus used in relation to a medical procedure. Specifically, any apparatus that contacts a patient during a medical procedure or therapy is contemplated herein as a medical device. Similarly, any apparatus that administers a compound or drug to a patient during a medical procedure or therapy is contemplated herein as a medical device. A medical device is “coated” when a collagen sheet becomes attached to the surface of the medical device. This attachment may be permanent or temporary. When temporary, the attachment may result in a controlled release of a drug or compound, such as, an antibiotic. In one embodiment, the medical device comprises a head implant. In one embodiment, the medical device comprises a neck implant. In one embodiment, the medical device is a titanium mandibular plate.

The term “collagen electrocompaction” as used herein refers to a method where a pure collagen solution may be compacted by inserting two planar carbon electrodes within a mold of desired size and shape. Using this technique, collagen may be compacted within a pre-designed lattice network. Akkus et al., “Electrochemical Processing Of Materials, Methods And Production” United States Patent Application Publication Number 2015/0376806 (herein incorporated by reference); and FIG. 4.

The term “suture” as used herein, refers to any a strand or fiber used to sew together living tissue.

The term “ELAC suture” as used herein, refers to a suture comprising a plurality of collagen threads that have undergone electrochemical alignment and compaction. An ELAC suture comprises chemical reaction sites capable of conjugationg a growth facor or attaching heparin.

The term “conventional suture” as used herein, refers to a suture that does not comprise collagen but is comprised of a plurality of threads that are generally made of materials such as nylon, polyester, silk or cotton. Such conventional sutures do not have chemical reaction sites capable of conjugating a growth factor or attaching heparin.

### BRIEF DESCRIPTION OF THE FIGURES

FIG. 1 presents one example of a collagen film encasing a medical device, for instance a titanium mandibular plate.

FIG. 2 presents exemplary data showing a proportional dose-dependence of heparinization as a function of collagen:heparin molar ratio.

FIG. 3 presents exemplary data showing the results of a diffusion assay against P. aeruginosa demonstrating a dose-dependent bactericidal activity of heparin crosslinked collagen sheets and no bactericidal activity of collagen-gentamicin sheets (control).

FIG. 4 presents one embodiment of an electrocompaction method to create a collagen sheet from a collagen solution.

FIG. 5 presents exemplary data of a disc bacterial diffusion inhibition assay as described in Example VII.

FIG. 6 presents exemplary data of a disc bacterial diffusion inhibition assay as described in Example VII.

FIG. 7 presents exemplary data of a disc moxifloxacin diffusion inhibition assay as described in Example VIII.

FIG. 8 presents exemplary data of a disc moxifloxacin diffusion inhibition assay as described in Example VIII.

FIG. 9 presents a schematic depiction of: a) the preparation of ELAC sutures with affinity bound platelet derived growth factor (PDGF-BB): (b) the principle of electrochemical process for preparing the aligned collagen threads (Cheng et al., “An electrochemical fabrication process for the assembly of anisotropically oriented collagen bundles,” Biomaterials 29 (2008) 3278-3288; (c) collagen threads go through three stages of chemical modifications of genipin crosslinking, Pet treatment, and heparin conjugation; (d) PGDF-BB solution is added on heparinized threads and retained by affinity binding; and (e) actual image of collagen threads following aforementioned chemical processing.

FIG. 10 presents an exemplary mechanical assessment of ELAC sutures during chemical modification stages: (a) ultimate tensile strength of ELAC sutures declines following Pet treatment and increase after heparinization in EDC/NHS. Failure strength and Young's modulus; and (b) the sutures in the final state do not differ significantly from those of genipin crosslinked sutures. Presence of heparin in EDC-NHS solution doesn't affect the crosslinking process of the collagen significantly. All significances reported at P<0.05.

FIG. 11 presents exemplary data that epitendinous ELAC sutures contributed to mechanical stabilization significantly beyond that is provided by the core suture: (a) A comparison between 6-0 standard nylon suture and aligned collagen suture; (b) A lacerated flexor tendon of chicken is sutured with aligned collagen suture to demonstrate the feasibility of suturing; (c) Ultimate failure load of lacerated tendon with epitendinous collagen sutures is greater than that of the tendon repaired with only core nylon suture, and, significantly less than the tendon repaired by the epitendinous 6-0 nylon sutures; (d) Typical load-displacement curves of lacerated tendons sutured under different conditions (Scale bar: 10 mm).

FIG. 12 presents exemplary data that swelling ratios of the ELAC sutures showed 52% increase after Pet process from 325% to 498% (P<0.05).

FIG. 13 presents exemplary data showing that the amount of heparin crosslinked to aligned collagen sutures does not affect proliferation of cells; a) The amount of heparin crosslinked to ELAC sutures can be controlled in a range from 0.19±0.02 mg/cm² of collagen to 1.32±0.20 mg/cm² by increasing the amount heparin from 1 mg/mL to 10 mg/mL in EDC-NHS solution: (b) Quantification of the cell number on samples with different amounts of heparin; and (c) typical DAPI stained images of cell nuclei demonstrated that cell proliferation was not affected significantly by the amount of heparin crosslinked to ELAC sutures (P<0.05, Scale bar: 100 μm).

FIG. 14 presents exemplary data showing that a PDGF-BB release timeline was delayed with increasing amount of heparin conjugation from 0.19±0.02 mg/cm² to 1.32±0.2 mg/cm² of aligned collagen sutures.

FIG. 15 presents exemplary data showing that cell morphologies visualized by Alexa-fluor staining of: a) cellular actin filaments demonstrated round and isotropic morphologies with no directional elongation on collagen gel; whereas; b) cellular actiin filaments on a collagen suture with affinity bound PDGF-BB; and c) cellular actin filaments on a collagen suture withou affinity bound PDGF-BB demonstrated a higher unidirectional elongation and anisotropic morphology; d) DNA quantification showing higher proliferation rate for cells on collagen suture with affinity bound PDGF-BB than cells on collagen suture without growth factor and collagen gel; and e) Alamar blue assay showing similar metabolic activity with no significant difference for cells in all three groups at day 1 and 7. (P<0.05, Scale bar: 50 lm).

FIG. 16 presents exemplary data of H&E staining after 3 weeks of culture showing; (c) a cell layer on aligned collagen sutures with affinity bound PDGF-BB; b) aligned collagen sutures without growth factor; and a) random collagen gel. Higher magnification images of H&E stained samples showed the differences in cell layer thicknesses in all three groups. Cell layer thickness on collagen suture with affinity bound PDGF-BB is greater than cells on collagen suture without growth factor and also the collagen gel. (Scale bars: 50 lm).

FIG. 17 presents exemplary data using Masson's trichrome staining of samples from all three groups of FIG. 16 demonstrating that de novo collagen was present in all groups after three weeks of cell culture. (a-c) The blue stain indicating the amount of deposited collagen by cells; and d) quantification of collagen amount that is released to the culture medium by the sircol assay which demonstrated that the amount of cell synthesized soluble collagen was significantly higher on aligned collagen sutures with affinity bound PDGF-BB compared to aligned collagen sutures without growth factor and random collagen gel. (P<0.05, Scale bar: 20 μm).

FIG. 18 presents exemplary data of tensile tests on collagen suture with affinity bound PDGF-BB showing that after 3 weeks of cell culture reduces the tensile strength and Young's modulus of the collagen suture significantly by 34% and 42%, respectively. (P<0.05).

FIG. 19 presents exemplary real time-PCR results showing significantly higher upregulation of tendon-related markers of scleraxis, tenomodulin, collagen I, and COMP on ELAC threads with and without PDGF-BB. However, the PCR results of day 21 demonstrated that release of PDGF-BB form ELAC threads down-regulated tendon related markers' expression moderately compared to collagen thread without PDGF-BB (d). (P<0.05).

FIG. 20 presents two embodiments of crosslinked ELAC threads.
A: Crosslinked with EDC/NHS
B: Crosslinked with genipin.

FIG. 21 presents exemplary data for failure loads for using low (1:10:25), medium (1:20:50) and high (1:100:250) molar ratios of collagen to EDC and NHS. Connecting bars indicated significant differences (p<0.05).

FIG. 22 presents exemplary data for modulus values of ELAC threads crosslinked using low (1:10:25), medium (1:20:50) and high (1:100:250) molar ratios of collagen to EDC and NHS. Connecting bars indicated significant differences (p<0.05).

FIG. 23 presents exemplary data for strain to failure of ELAC threads using low (1:10:25), medium (1:20:50) and high (1:100:250) molar ratios of collagen to EDC and NHS. Connecting bars indicated significant differences (p<0.05).

FIG. 24 presents exemplary data for mean failure load of ELAC threads crosslinked in varying percentages of ethanol in water or MES buffer. Connecting bars indicate significant differences between threads crosslinked with the same percentage of ethanol in different co-solvents. A single star indicates no significant difference in failure load from threads crosslinked in 2% genipin (α=0.05). Two stars indicate significantly greater failure loads than those of threads crosslinked in 2% genipin (α=0.05).

FIG. 25 presents exemplary data for modulus values for ELAC threads crosslinked in variable percentages of ethanol and water or MES buffer. A single star indicates mean modulus values are significantly greater than modulus values for ELAC threads crosslinked in 2% genipin. Three stars indicate modululs values that significantly less than those for ELAC threads crosslinked in 2% genipin. No symbol indicates modulus values are not significantly different from ELAC threads crosslinked in 2% genipin (α=0.05).

FIG. 26 presents exemplary data for strain to failure of ELAC threads crosslinked in variable percentages of ethanol in water and MES buffer. Three stars indicate a mean strain to failure that is significantly less than that of ELAC threads crosslinked in 2% genipin (α=0.05).

FIG. 27 presents exemplary data for mean failure loads for different crosslinking times and repetitions. Three stars indicate that the mean failure load is significantly less than the mean failure load of ELAC threads crosslinked for two hours and then overnight.

FIG. 28 presents exemplary data for mean modulus values for different crosslinking times and repetitions. Three stars indicate that that modulus values are significantly less modulus values of ELAC threads crosslinked for two hours and then overnight (α=0.05).

### DETAILED DESCRIPTION OF THE INVENTION

This invention is related to the field of medical device implants. In particular, the invention is related to sutures and antibacterial coatings that comprise collagen. In particular, some of the collagen-based coatings prevent and control of bacterial infections that result from the use of medical device implants (e.g., head and neck implants). One method of preventing post-medical device implant bacterial infections comprises using a medical device coated with a biologic antimicrobial coating. For example, one such coating comprises a covalently crosslinked heparin-collagen sheet.

I. Bacterial Infection from Medical Device Implants

Implanted medical devices provide a surface on which many species of bacteria are able to adhere and produce a biofilm matrix. Biofilms are highly resistant to antibiotic penetrance and the host immune system and have been shown to be a common cause of persistent infections

Bacterial infection is believed to be one of the most common and devastating complications associated with medical implants. A recent review of nearly 3000 patients with facial fracture requiring plate fixation showed that infection was the most common complication and the most common reason for hardware removal.

These data show that the majority of hardware removals were precipitated by infection (boxed area). Rosa et al. “Review of Maxillofacial Hardware Complications and Indications for Salvage.” Craniomaxillofacial Trauma and Reconstruction 9.02 (2016): 134-140. The detailed data for this study is listed in Tables 2 and 3.

TABLE 2
Description And Frequency Of All Complications At Recipient Site

| | | No. (%), |
|---|---|---|
| Category | Complication | N = 65 |
| Perioperative | Flap failure with revision | 3 (5) |
| complication | Partial failure with revision | 4 (6) |
| | Infections | 10 (15) |
| | Hematoma | 2 (3) |
| Late complication | Full revision | 2 (3) |
| | Partial revision | 5 (8) |
| | Cellulitis | 5 (8) |
| | Fistulas | 7 (11) |
| | Osteomyelitis | 9 (14) |
| | Hardware issues | 10 (15) |
| | Nonunion mandible | 3 (5) |

TABLE 3
Analysis Of Outcomes Categories

| Outcome 1 | Outcome 2 | OR (95% CI) | |
|---|---|---|---|
| Perioperative complication | Early infection | 63.3 | (6.8-585.3) |
| | Late failure | 2.8 | (0.5-15.6) |
| | Late complication | 1.2 | (0.4-3.9) |
| Perioperative infection | Early revision | 1.9 | (0.3-11.8) |
| | Late complication | 4.8 | (1.3-18.3) |
| | ORN/osteomyelitis | 8.8 | (1.8-41.9) |
| | Late failure | 12.8 | (1.9-84.5) |

OR = odds ratio;
CI = confidence interval;
ORN = osteoradionecrosis.

These data show that bacterial infection was the most common perioperative complication and significantly worsened outcomes. Zender et al., “Etiologic causes of late osteocutaneous free flap failures in oral cavity cancer reconstruction.” The Laryngoscope 122.7 (2012): 1474-1479.

Prior attempts to increase the antibacterial properties of implantable hardware have met a number of limitations that include, but are not limited to, poorly controlled antibiotic release, limited chemical stability, local tissue toxicity, poor osteointegration, inhibition of osteoblastic activity. None of the prior art methods have entered standard practice in head and neck surgery due to these known limitations. For example, 10-15% of hardware placed for facial fracture reconstruction is ultimately removed, and the majority of those removals are related to infection.

Francel et al., “The fate of plates and screws after facial fracture reconstruction.” Plastic and reconstructive surgery 90.4 (1992): 568-573; Simchi et al. “Recent progress in inorganic and composite coatings with bactericidal capability for orthopaedic applications.” Nanomedicine: Nanotechnology, Biology and Medicine 7.1 (2011): 22-39; and Abubaker et al., “Postoperative antibiotic prophylaxis in mandibular fractures: A preliminary randomized, double-blind, and placebo-controlled clinical study.” Journal of oral and maxillofacial surgery 59.12 (2001): 1415-1419.

To date, two pathophysiological processes have been implicated in the infection of implants: local immune response suppression and biofilm formation. Implants provide a surface on which many species of bacteria are able to adhere and produce a biofilm matrix. These biofilms are highly resistant to both antibiotic penetrance and the host immune system. Furthermore, they are associated with increased antibiotic resistance. Given the tremendous burden of these infections, the projected increase in the use of implantable devices, and the growing problem of bacterial resistance, the development of strategies to prevent device-related infections is more pressing than ever.

As peri-prosthetic infection has been reporated as a complication associated with implant placement, it was reasoned that such infections can be largely subverted by development of antibacterial implants. For example, covalent coupling of vancomycin to titanium alloy prevents colonization by the Gram-positive pathogens, Staphylococcus aureus and Staphylococcus epidermidis. Some orthopedic devices, including permanent prosthesis anchors, and most dental implants are transcutaneous or transmucosal and can be prone to colonization by Gram-negative pathogens. Furthermore, covalent coupling of a broad-spectrum antibiotic, tetracycline (TET), to titanium surfaces (Ti-TET) retarded Gram-negative colonization. Ti-TET actively prevented colonization in the presence of bathing Escherichia coli, both by fluorescence microscopy and direct counting. Finally, the Ti-TET surface supported osteoblastic cell adhesion and proliferation over a 72-h period. Davidson et al., (2014). “Tetracycline tethered to titanium inhibits colonization by Gram-negative bacteria.” J Biomed Mater Res Part B: Appl Biomater.

As the use of implantable devices rise, some patients are unable to autonomously prevent formation of biofilm on implant surfaces. Suppression of the local peri-implant immune response may be a contributory factor. Substantial avascular scar tissue encountered during revision joint replacement surgery places these cases at an especially high risk of periprosthetic joint infection. One pathogenic event occuring in the process of biofilm formation is believed to be bacterial adhesion. Prevention of biomaterial-associated infections should be concurrently focused on at least two targets: inhibition of biofilm formation and minimizing local immune response suppression. Gallo et al., (2014) “Antibacterial surface treatment for orthopaedic implants.” Int J Mol Sci 15(8): 13849-13880.

From 2002 to 2006, more than 117,000 facial fractures were recorded in the U.S. National Trauma Database. These fractures are commonly treated with open reduction and internal fixation. Plate implants, while in place, facilitates successful bony union. However, when postoperative complications occur, the plates may require removal before bony union is completed. Indications for salvage versus removal of the maxillofacial hardware are not well defined. Complication rates from such fractures have been observed to vary from 6 to 8% by fracture and 6 to 13% by patient. When their data were combined, one assessment suggests that 50% of complications were treated with plate removal; this was consistent across the mandible, midface, and upper face. Some complications caused by loosening, nonunion, broken hardware, and severe/prolonged pain can be treated with removal, while other complications caused by exposures, deformities, and infections may be treated with salvage. Exposed plates can be treated with flaps, plates with deformities are treated with secondary procedures including hardware revision, and hardware infections were treated with antibiotics alone or in conjunction with soft-tissue debridement and/or tooth extraction. Rosa et al. (2016) “Review of Maxillofacial Hardware Complications and Indications for Salvage.” Craniomaxillofac Trauma Reconstr 9(2): 134-140.

Collagen-based biomaterials are reported to be a viable option for tendon reconstruction and repair. However, the weak mechanical strength of collagen constructs is a major limitation. Highly oriented electrochemically aligned collagen (ELAC) threads have been reported having mechanical properties converging on those of the natural tendon. For example, an in vivo response of rabbit patellar tendon (PT) to braided ELAC bioscaffolds was assessed. Rabbit PTs were incised longitudinally and the ELAC bioscaffold was inlaid in one limb along the length of the tendon. The contralateral limb served as the sham-operated control. Rabbits were euthanized at 4 or 8 months postoperatively. High-resolution radiographs revealed the absence of ectopic bone formation around the bioscaffolds. Four months post-implantation, the histological sections showed that the ELAC bioscaffold underwent limited degradation and was associated with a low-grade granulomatous inflammation. Additionally, quantitative histology revealed that the cross-sectional areas of PTs with the ELAC bioscaffold were 29% larger compared with the controls. Furthermore, ELAC-treated PTs were significantly stiffer compared with the controls. The volume fraction of the tendon fascicle increased in the ELAC-treated PT compared with the controls. By 8 months, the ELAC bioscaffold was mostly absorbed and the enlargement in the area of tendons with implants subsided along with the resolution of the granulomatous inflammation. This study suggests that ELAC is biocompatible and biodegradable and has the potential to be used as a biomaterial for tendon tissue engineering applications. Kishore et al., (2012) “In vivo response to electrochemically aligned collagen bioscaffolds.” J Biomed Mater Res B Appl Biomater 100(2): 400-408.

Recent developments of inorganic and organic-inorganic composite coatings for orthopedic implants, providing an interface with living tissue and with potential for drug delivery to combat infections have been recently reviewed. Conventional systemic delivery of drugs is an inefficient procedure that may cause toxicity and may require a patient's hospitalization for monitoring. Local delivery of antibiotics and other bioactive molecules maximizes their effect where they are required, reduces potential systemic toxicity and increases timeliness and cost efficiency. In addition, local delivery has broad applications in combating infection-related diseases. Polymeric coatings may present some disadvantages. These disadvantages include, but are not limited to, limited chemical stability, local inflammatory reactions, uncontrolled drug-release kinetics, late thrombosis and/or restenosis. As a result, embedding of bioactive compounds and biomolecules within inorganic coatings (bioceramics, bioactive glasses) is attracting significant attention. Recently, nanoceramics have attracted interest because surface nanostructuring allows for improved cellular adhesion, enhances osteoblast proliferation and differentiation, and increases biomineralization. Organic-inorganic composite coatings, which combine biopolymers and bioactive ceramics that mimick bone structure to induce biomineralization, with the addition of biomolecules, represent alternative systems and ideal materials for “smart” implants. Simchi et al., (2011) “Recent progress in inorganic and composite coatings with bactericidal capability for orthopaedic applications.” Nanomedicine 7(1): 22-39.

Isoelectric focusing (IEF) of type-I collagen molecules is a technology can produce dense and aligned collagen-based biomaterials. Even so, the forces and mechanisms during IEF of collagen molecules in carrier ampholyte-free environments remain unknown. Theoretical frameworks describing the congregation of collagen molecules along the isoelectric point (pI) have been put forward. For example, a single molecule was modeled as a rod-like particle, distributed homogeneously between parallel electrodes. Upon application of electrical current, molecules migrated to the pI. The results showed that self-aggregation of collagen molecules along the pI occurred due to formation of a non-linear pH gradient that rendered the anodic side acidic, and the cathodic side basic. This pH profile and the amphoteric nature of collagen resulted in positively charged molecules at the anode and negatively charged molecules at the cathode. Therefore, repulsive electrostatic forces aided self-aggregation of molecules along the pI. The model could effectively validate the pI of collagen, the pI location, and predict that the instantaneous velocity acting on a molecule at the anode was higher than those velocities at the cathode. This fundamental information represents a baseline theory for the production of biomaterials to engineer soft tissues. Uquillas et al., (2012) “Modeling the electromobility of type-I collagen molecules in the electrochemical fabrication of dense and aligned tissue constructs.” Ann Biomed Eng 40(8): 1641-1653.

Diabetic foot infections are frequently polymicrobial in nature. Notabley, a lower tissue concentration of systemically administered antibiotics have been observed in diabetic patients as opposed to non-diabetic patients. One suggested alternative compound is Collatamp®; EG (Syntacoll GmbH Saal/Donau, Germany) which is a bioabsorbable, gentamicin impregnated collagen sponge and used for local treatment. For example, a gentamicin-collagen sponge was applied to a post-surgical wound after minor amputations in diabetic patients. Fifty diabetic patients indicated for minor amputation were pre-operatively randomised into two groups. Twenty-five patients in group A were treated with gentamicin impregnated collagen sponge applied into wound peri-operatively while 25 patients in group B had minor amputation without gentamicin sponge. There was no significant difference in the demographic data, procedures performed, diabetes duration and peripheral vascular disease severity between the groups. The median glycosylated haemoglobin was 6.0% (range: 4.6-9.5%) in group A and 6.2% (range: 4.0-8.4%) in control group B (non-significant). Median TcPO2 level was 44 (range: 13-67) in group A and 48 (range: 11-69) in control group B (non-significant). The median of wound healing duration in group A was 3.0 weeks (range: 1.7-17.1 weeks) compared to 4.9 weeks (range: 2.6-20.0 weeks) in control group B. This was with a statistically significant difference (p<0.05). Consequently, the application of gentamicin impregnated collagen sponge shortened wound healing duration after minor amputations in diabetic patients by almost 2 weeks. Varga et al., (2014) “Application of gentamicin-collagen sponge shortened wound healing time after minor amputations in diabetic patients—a prospective, randomised trial.” Arch Med Sci 10(2): 283-287.

The effects of Gentacoll® implants was also investigated for healing in patients (n=44) undergoing modified radical mastectomy and axillary dissection. In Group I, the Gentacoll® group (n=22), underwent surgery followed by insertion of 10×10×0.5 cm Gentacoll® implants (280 mg collagen sponge plus 200 mg gentamicin sulphate) into the axillary area and under the flap area of the breast before wound closure. In Group II, the control group (n=22), underwent surgery without the application of Gentacoll®. Neither group received oral or parenteral post-operative antibiotic therapy. Outcome measures included wound infection, seroma formation, total drainage volumes, drain removal time and duration of hospital stay. Post-operative infection rate, seroma formation, drainage volumes and duration of hospital stay were significantly reduced in the Gentacoll® group compared with the control group. In conclusion, the application of Gentacoll® significantly improved post-operative outcomes in patients undergoing modified radical mastectomy. Yetim et al., (2010) “Effect of local gentamicin application on healing and wound infection in patients with modified radical mastectomy: a prospective randomized study.” J Int Med Res 38(4): 1442-1447.

Infection is usually defined as a homeostatic imbalance between the host tissue and the presence of microorganisms. It is associated with a large variety of wound occurrences ranging from traumatic skin tears and burns to chronic ulcers and complications following surgery and device implantations. If the wound setting manages to overcome the microorganism invasion by a sufficient immune response then the wound should heal. If not, the formation of an infection can seriously limit the wound healing process. Evidence of increasing bacterial resistance is on the rise, and complications associated with infections are therefore expected to increase. Generally, treatments of various types of wound infections decrease the bacterial load in the wound to a level that enables wound healing processes to take place. Conventional systemic delivery of antibiotics entails poor penetration into ischemic and necrotic tissue and can cause systemic toxicity with associated renal and liver complications, which result in a need for hospitalization for monitoring. Alternative local delivery of antibiotics by either topical administration or by a delivery device may enable the maintenance of a high local antibiotic concentration for an extended duration of release without exceeding systemic toxicity. Various approaches for local prevention of bacterial infections based on antibiotic-eluting medical devices have been identified. These devices include, but are not limited to, bone cements, fillers, coatings for orthopedic applications, wound dressings based on synthetic or natural polymers, intravascular devices, vascular grafts and/or periodontal devices. Alternatively, composite drug-eluting fibers and structured drug-eluting films, which are designed to be used as basic elements of various devices have also been suggested. Zilberman et al., (2008) “Antibiotic-eluting medical devices for various applications.” Journal of Controlled Release 130(3): 202-215.

In summary, there has been growing interest in localized infection prophylaxis. As discussed above, a variety of methods employed fall into at least two categories: the incorporation of antibacterial agents into the surface materials of devices and the introduction of local antibiotic delivery systems. Though some of these methods have been shown to have efficacy, they are limited by poorly controlled antibiotic release, limited chemical stability, local tissue toxicity, poor osteointegration, and/or inhibition of osteoblastic activity.

To address these shortcomings, some embodiments of the present invention contemplate a novel biologic antimicrobial coating for medical implants. In one embodiment, an antimicrobial coating comprises a modified electrochemical compaction of type-1 collagen molecules. Although it is not necessary to understand the mechanism of an invention, it is believed that such electrochemical compaction creates dense sheets comprising covalently-bonded heparin and electrostatically-associated gentamicin. These sheets are mechanically suited for encasing medical devices prior to implantation. For example, such sheets may be on the order of approximately 100 microns thick, flexible, robust, and can be cut to size. Although it is not necessary to understand the mechanism of an invention, it is beleved that the collagen sheets described herein have a potential to prevent infection in at least a two-fold manner by: i) acting as a physical barrier to biofilm formation; and ii) providing modulated, affinity-based local release of gentamicin.

II. Biologic Antibacterial Coatings

It is contemplated that the presently disclosed composition is active against most pathogens commonly implicated as a result of the implantation of medical devices (i.e., for example, post-operative head and neck infections). The presently disclosed heparin-collagen sheet offers many advantages over commonly used antibacterial coatings, including but not limited to: i) the sheet can be applied to existing hardware and is not limited to a manufacturing process; ii) minimal impact on surgery duration; iii) enhances wound healing (Sixta et al. 2014); iv) can be produced quickly and inexpensively, and v) has the potential to prevent infection by acting as a physical barrier to biofilm formation and by providing sustained local antibiotic delivery as the collagen film is resorbed in the wound healing process. Other advanatages also include, but are not limited to: i) maximizes local antibiotic delivery; ii) provides several weeks of antimicrobial protection; iii) acts as a physical barrier to bacterial attachment; iv) does not alter established surgical techniques and v) is biocompatible. Varga et al. (2014). “Application of gentamicin-collagen sponge shortened wound healing time after minor amputations in diabetic patients—a prospective, randomised trial.” Arch Med Sci 10(2): 283-287.

In one embodiment, the present invention contemplates embodiments including, but not limited to: i) pure collagen sheets containing antimicrobial therapeutics; ii) durable thin sheets that can be cut to size and used to encase hardware prior to implantation; or iii) resorbable collagen sheets such that incorporated compounds (i.e., for example, antibiotics) are released over time. For example, in one embodiment, heparin-collagen sheets could be cut to size and used to encase implantable hardware, providing sustained local antibiotic delivery over the initial weeks of wound healing.

In one embodiment, the present invention contemplates a method of collagen compaction to create pure collagen sheets impregnated with antibiotics. Heparin may then be covalently crosslinked to collagen using 1-(3-(dimethylamino)propyl)-3-ethylcarbodiimidehydrochloride (EDC) and N-hydroxysulfosuccinimide (NHS), and optionally a gentamicin solution allows for electrostatic association of gentamicin to heparin.

Subsequently, a quantitative demonstration of heparin bonding may be performed using a dimethyl methylene blue spectrophotometric assay. This assay quantitative demonstrates that heparin bonding occurs using a DMMB spectrophotometric assay for sulfated GAGs, where heparinized films were dissolved in 1 M HCl at 85° C. and DMMB assay was used to measure the concentration of heparin in the solution.

A functional test of antibacterial sheet may then be performed, for example, by using bacterial diffusion assays against Pseudomonas aeruginosa and/or Staphylococcus aureus. This functional test is generally a bacterial diffusion assay in which small discs of collagen sheets are plated onto agar with the most commonly implicated bacteria in head and neck surgical site infections. The test groups are gent-heparin-collagen sheets and the control group is collagen sheets that have undergone gentamicin incubation but no heparinization. Radii of zones of inhibition are measured at 24-hour time points over 5-7 days.

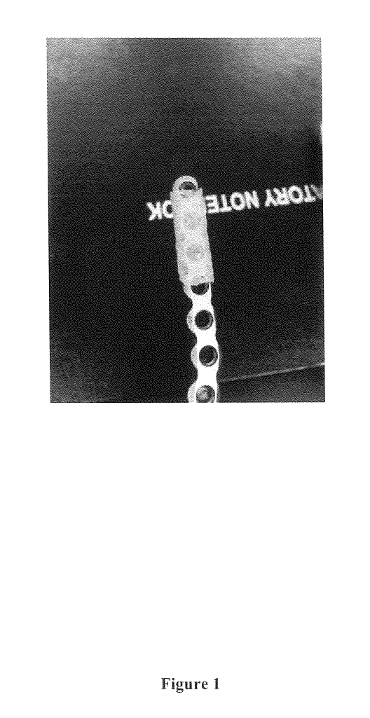

Collagen sheets were fabricated with mechanical qualities suitable for encasing implantable hardware, including flexibility, robustness, and thickness of 100 microns. For example, a collagen sheet was used to encase a titanium mandibular plate. FIG. 1.

Qualitative and quantitative confirmation of heparin covalent crosslinking to collagen was performed using DMMB staining assays for sulfated glycosaminoglycans. Example II.

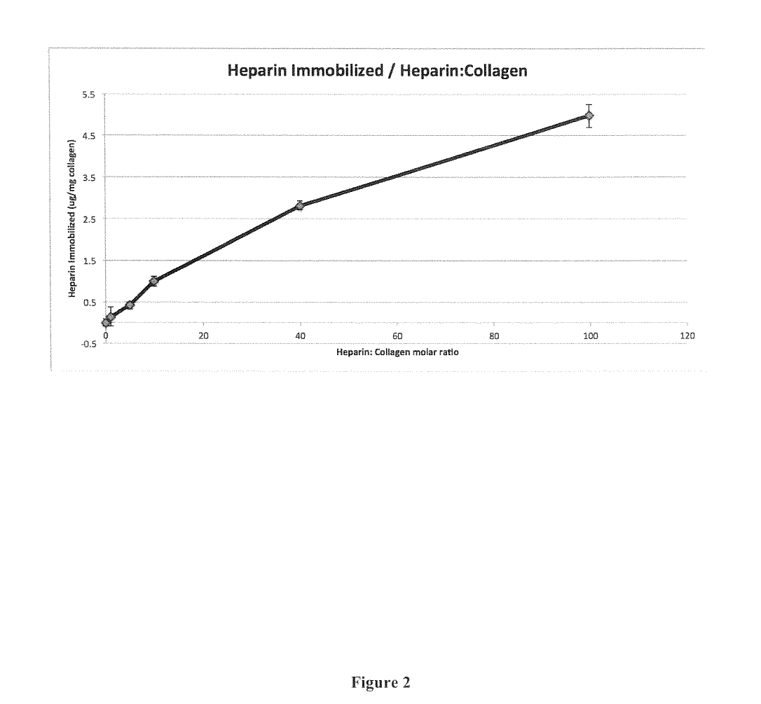

Collagen films created at various heparin:collagen molar ratios have demonstrated a dose-dependent increase in the amount of heparin that can be crosslinked to the collagen. The data demonstrate an absence of DMMB staining of collagen-only sheets and progressive increase of DMMB stain intensity of heparin-collagen films in proportion with increased heparin concentration. FIG. 2.

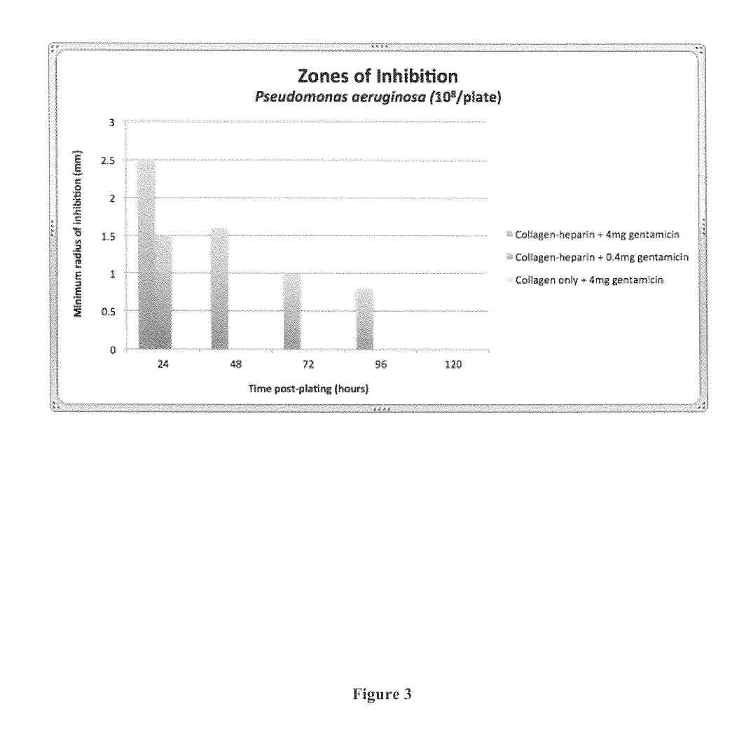

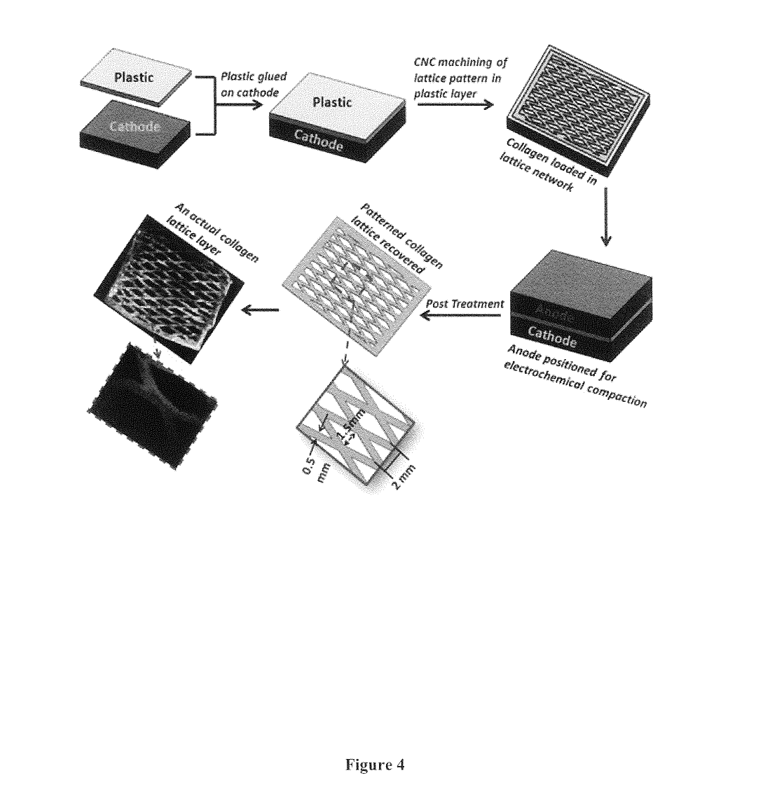

Antibacterial diffusion assays against Pseudomonas aeruginosa of a biologic antibacterial composition comprising a crosslinked heparin collagen sheet have demonstrated dose-dependent bactericidal activity. FIG. 3 (X: time post-plating; Y: minimum radius of inhibition measured) and Example III. The blue bars represent the test group (gentamicin-heparin-collagen sheets) with highest concentration of gentamicin and the red bars represent a test group with lower concentration of gentamicin. The control group (gentamicin-collagen sheets; e.g., non-heparinized), produced no zones of inhibition. The data show that the test collagen sheets with highest heparin-gentamicin loading produced largest zones of inhibition and demonstrate dose-dependent bactericidal activity.

It is contemplated that by reducing the rate of infection from medical device implants by the use of the heparin-collagen sheets will improve medical treatment results including, but not limited to, i) faster recovery times; ii) reduced patient morbidity; iii) improved functional and cosmetic outcomes; and iv) reduced medical costs associated with hardware infection. Given the undeniable medical and societal burden of these infections, the growing problem of bacterial resistance, and the projected increase in the use of implantable devices, the presently disclosed antibacterial sheet provides a surprising and unpredictable option for the use in medical device surgery.

III. Collagen/Heparin Sutures for Drug Delivery

Suturing is believed to be the current standard of repair for lacerated flexor tendons. Past studies focused on delivering growth factors to the repair site by incorporating growth factors to nylon sutures which are commonly used in the repair procedure. However, conjugation of growth factors to nylon or other synthetic sutures is not straightforward. Collagen holds promise as a suture material by way of providing chemical sites for conjugation of growth factors. On the other hand, collagen also needs to be reconstituted as a mechanically robust thread that can be sutured.

In one embodiment, the present invention contemplates a reconstituted collagen solution for creating suturable collagen sutures by using linear electrochemical compaction. For example, prolonged release of platelet derived growth factor-BB (PDGF-BB) was achieved by covalent bonding of heparin to a saturable collagen suture. Tensile mechanical tests of collagen sutures made before and after chemical modification indicated that the strength of sutures following chemical conjugation stages was not compromised. Strength of lacerated tendons sutured with epitendinous collagen sutures (11.2±0.7 N) was comparable to that of standard nylon sutures (14.9±2.9 N). Heparin conjugation of collagen sutures didn't affect viability and proliferation of tendon-derived cells and prolonged the PDGF-BB release up to 15 days. Proliferation of cells seeded on PDGF-BB incorporated collagen sutures was about 50% greater than those seeded on plain collagen sutures. Collagen that is released into the media by the cells increased by 120% under the effects of PDGF-BB and collagen production by cells was detectable by histology twenty-one (21) days after suture placement. Addition of PDGF-BB to collagen sutures resulted in a moderate decline in the expression of the tendon-associated markers scleraxis, collagen I, tenomodulin, and COMP; however, expression levels were still greater than the cells seeded on collagen gel. The data indicate that the effects of PDGF-BB on tendon-derived cells mainly occur through increased cell proliferation and that longer term studies are needed to confirm whether this proliferation outweighs the moderate reduction in the expression of tendon-associated genes.

A mechanically robust collagen suture was fabricated via linear electrocompaction and conjugated with heparin for prolonged delivery of PDFG-BB. Sustained delivery of the PDGF-BB improved the proliferation of tendon derived cells substantially at the expense of a moderate downregulation of tenogenic markers. The collagen sutures described herein were as functionally applicable as epitendinous sutures when applied to chicken flexor tendons in vitro. Overall, electrocompacted collagen sutures holds a potential to improve repair outcome in flexor tendon surgeries by improving cellularity and collagen production through delivery of the PDGF-BB. The bioinductive suture concept can be applied to deliver other growth factors for a wide-array of applications.

A. Conventional Suture Technology

More than 30 million tendon and ligament injuries are reported globally every year. The most common treatment of lacerated or ruptured tendons is suture based repair. This treatment is generally attained by a mechanical load-bearing core suture. Kleinert et al., “Primary repair of flexor tendons” The Orthopedic Clinics of North America 4:865-876 (1973). Epitendinous sutures are used to oppose damaged ends and to ease the gliding of the injured tendon. However, tendons are relatively poorly vascularized and healing is a slow process which may result in fibrosis with inferior mechanics and function. Therefore, novel strategies are needed to accelerate tendon healing and to improve the quality of regeneration.

Growth factors are believed to be powerful regulators of biological function. The patterns of natural expression of platelet derived growth factor (PDGF-BB), basic fibroblast growth factor (b-FGF), transforming growth factor-β (TGF-β1), and vascular endothelial growth factor (VEGF) vary dramatically over time during tendon healing. According to recent studies, growth factor supplementation strategies have shown significant functional value in the context of tendon tissue regeneration. Molloy et al., “The roles of growth factors in tendon and ligament healing” Sports Med. 33:381-394 (2003); Thomopoulos et al., “Effect of several growth factors on canine flexor tendon fibroblast proliferation and collagen synthesis in vitro” J. Hand Surg. Am. 30:441-447 (2005); and Costa et al., “Tissue engineering of flexor tendons: optimization of tenocyte proliferation using growth factor supplementation” Tissue Eng. 12:1937-1943 (2006).

Individually, PDGF-BB and insulin-like growth factor 1 (IGF-1) have been shown to alter proliferation, biological activities and collagen synthesis by tendon cells. Yoshikawa et al., “Dose-related cellular effects of platelet derived growth factor-BB differ in various types of rabbit tendons in vitro” Acta Orthop. Scand. 72:287-292 (2001); and Abrahamsson et al., “Recombinant human insulin-like growth factor-I stimulates in vitro matrix synthesis and cell proliferation in rabbit flexor tendon” J. Orthop. Res. 9:495-502 (1991). The recent past has seen the delivery of potent growth factors to the injury site to expedite tendon healing. Previous studies have applied growth factors to the injury site via local injection. Zhang et al., “Effect of vascular endothelial growth factor on rat Achilles tendon healing” Plast. Reconstr. Surg. 112:1613-1619 (2003).

Although local injection is minimally-invasive, the short half-life of the growth factor (e.g., a few minutes for PDGF-BB) limits their therapeutic benefit. Several studies have exploited already existing sutures at repair site as vehicles for growth factor delivery. Bowen-Pope et al., “Platelet-derived growth factor in vivo: levels, activity, and rate of clearance” Blood 64:458-469 (1984); Dines et al., “In vitro analysis of an rhGDF-5 suture coating process and the effects of rhGDF-5 on rat tendon fibroblasts” Growth Factors 29:1-7 (2011); Henn et al., “Augmentation of zone II flexor tendon repair using growth differentiation factor 5 in a rabbit model” J. Hand Surg. Am. 35A:1825-1832 (2010); and Dines et al., “The effect of growth differentiation factor-5-coated sutures on tendon repair in a rat model” J. Shoulder Elbow Surg. 16:S215-S221 (2007). Utilization of sutures may drive the repair to the inner continuum of the tendon stumps and expedite healing with increased repair strength. Past studies employed coating of standard synthetic polymeric sutures with growth factor doped gelatin or growth factor added scaffolds (generally fibrin). Thomopoulos et al., “The effects of exogenous basic fibroblast growth factor on intrasynovial flexor tendon healing in a canine model” J. Bone Joint Surg. Am. 92:2285-2293 (2010); and Thomopoulos et al., “PDGF-BB released in tendon repair using a novel delivery system promotes cell proliferation and collagen remodeling” J. Orthop. Res. 25:1358-1368 (2007).

These modifications are reported to achieve some success in the flexor tendon repair. Gelatin coating degrades fast and it is stripped while passing the suture. Fibrin scaffolds are weak and may be limited in terms of load bearing applications. Xenogeneic sutures are available on the market such as those prepared from the gut (Ethicon, Inc.). Dann, Ethicon Wound Closure Manual, Ethicon Inc., PO Box 151, Somerville, N.J. 08876-0151, 2005; and Greenberg et al., “Advances in suture material for obstetric and gynecologic surgery” Rev. Obstet. Gynecol. 2:146-158 (2009). Surgical gut suture may induce granuloma formation. Truhlsen et al., “The extruded collagen suture: tissue reaction and absorption” Arch. Ophthalmol. 74:371-374 (1965).

Other researchers developed and used syringe extruded collagen and fibrin threads for delivering stem cells to cardiac tissues. Guyette et al., “A novel suture-based method for efficient transplantation of stem cells” J. Biomed. Mater. Res. A 101:809-818 (2013); and Proulx et al., “Fibrin microthreads support mesenchymal stem cell growth while maintaining differentiation potential” J. Biomed. Mater. Res. A 96:301-312 (2011).

The bioactive suture concept needs to be improved to develop mechanically robust suture materials that can retain growth factors. Collagen is conducive to cell adhesion, motility, proliferation and/or differentiation. Gey et al., “Long-term growth of chicken fibroblasts on a collagen substrate” Exp. Cell Res. 84:63-71 (1974); Gospodarowicz et al., “Do plasma and serum have different abilities to promote cell growth?” Proc Natl. Acad. Sci. USA 77:2726-2730 (1980); Hauschka et al., “The influence of collagen on the development of muscle clones” Proc. Natl. Acad. Sci. USA 55:119-126 (1966); Kosher et al., “Stimulation of in vitro somite chondrogenesis by procollagen and collagen” Nature 258:327-330 (1975); Lash et al., “Somite chondrogenesis in vitro. Stimulation by exogenous extracellular matrix components” Dev. Biol. 66:151-171 (1978); and Meier et al., “Control of corneal differentiation by extracellular materials. Collagen as a promoter and stabilizer of epithelial stroma production” Dev. Biol. 38:249-270 (1974).

It is believed that collagen can present chemical sites for conjugation of growth factors. For example, load-bearing growth factor conjugated pure collagen sutures have not been used for repair of lacerated tendons. Nonetheless, electrochemical compaction and alignment of collagen molecules as described herein provides load-bearing collagen sutures. Such compaction and alignment has also been shown to induce tenogenic differentiation of mesenchymal stem cells (MSCs). Younesi et al., “Tenogenic induction of human MSCs by anisotropically aligned collagen biotextiles” Adv. Funct. Mater. 24:5762-5770 (2014); Uquillas et al., “Genipin crosslinking elevates the strength of electrochemically aligned collagen to the level of tendons” J. Mech. Behav. Biomed. Mater. 15:176-189 (2012); Islam et al., “Computer aided biomanufacturing of mechanically robust pure collagen meshes with controlled macroporosity” Biofabrication 7:035005 (2015); Younesi et al., “Fabrication of compositionally and topographically complex robust tissue forms by 3D electrochemical compaction of collagen” Biofabrication 7:035001 (2015); and Kishore et al., “Tenogenic differentiation of human MSCs induced by the topography of electrochemically aligned collagen threads” Biomaterials 33:2137-2144 (2012).

B. Electrochemically Aligned/Compacted Collagen Suture Technology

As discussed above, electrochemically aligned and compacted collagen (ELAC) hold promise as an epitendinous suture for repair of lacerated tendons. Electrocompaction of collagen to produce aligned threads has been previously published. Cheng et al., “An electrochemical fabrication process for the assembly of anisotropically oriented collagen bundles,” Biomaterials. vol. 29, no. 22, pp. 3278-3288, 2008. Briefly, acidic, monomeric collagen (Collagen Solutions Inc, California) is diluted to a concentration of 3 mg/mL using deionized water and then dialyzed overnight against deionized water. The dialyzed collagen solution is then applied between two wire electrodes and compacted into highly aligned collagen threads using isoelectric focusing.

Isoelectric focusing is based on the principle that the overall charge on a protein is a function of the pH of its surroundings. The pH at which the net charge of a protein is neutral is known as the isoelectric point. Molecules above and below this pH will be negatively and positively charged respectively, and as such, will be pulled towards the oppositely charge electrode. As the molecules move through the increasing or decreasing pH gradient their overall charge will decrease until they reach the isoelectric point. Hydrolysis of water at either electrode creates a pH gradient for collagen molecules to travel along. The isoelectric point of collagen is around a pH of 8 so alignment of collagen molecules takes place close to the negatively charged cathode. Uquillas J, “Modeling the electromobility of type-I collagen molecules in the electrochemical fabrication of dense and,” Ann. Biomed. Eng., vol. 40, no. 8, pp. 1641-1653, 2012.

ELAC threads can be collected in finite lengths using parallel wires acting as the anode and cathode to create an electrochemical cell. ELAC threads can also be collected in continuous lengths using an “infinite” electrode set-up in which parallel wires are embedded into a channel within a rotating circular wheel.

In one embodiment, the present invention contemplates a tenogenic suturable ELAC suture comprising a growth factor. In one embodiment, the growth factor is PDGF-BB. In one embodiment, the ELAC surture further comprises a plurality of conjugated heparin molecules. In one embodiment, the growth factor attaches to the heparin molecule. In one embodiment, the growth factor/heparin attachment is by affinity bonding.

The effects of the growth factor delivery on proliferation, metabolic activities, collagen production and expression of tendon-related genes by tendon-derived cells were investigated. The data presented herein demonstrate that ELAC sutures are functional as epitendinous sutures and have a biomechanical attachment strength comparable to standard nylon sutures.

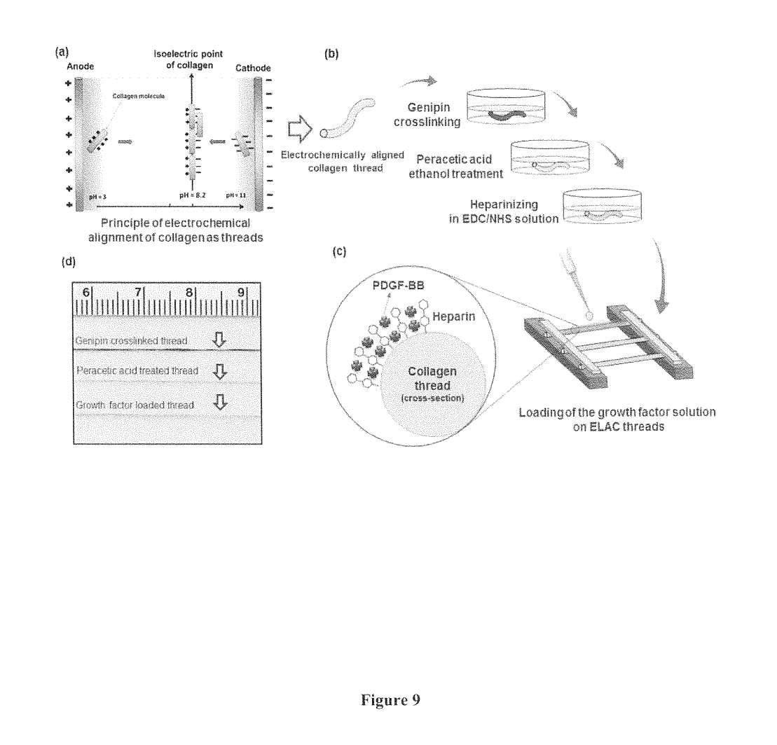

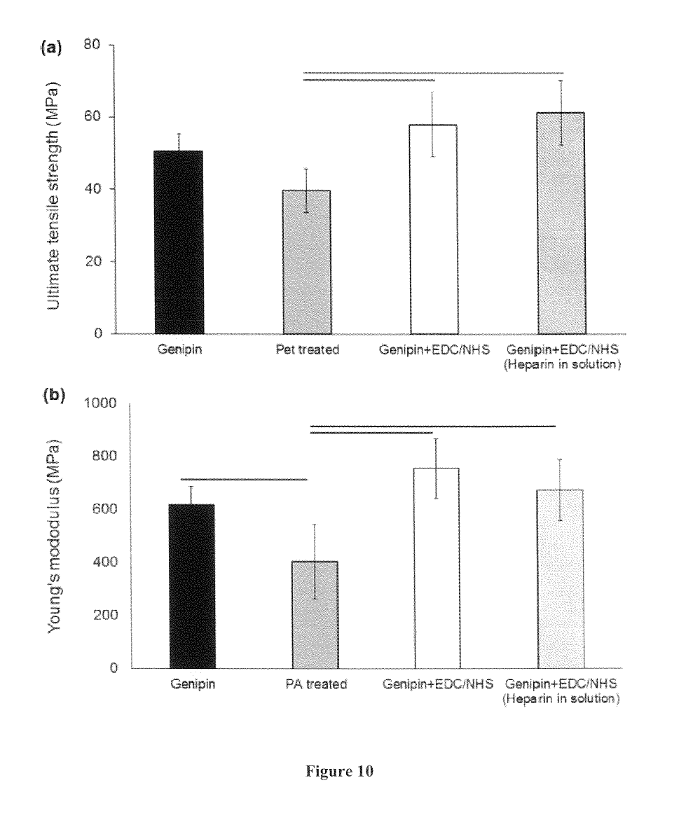

In one embodiment, the present invention contemplates a suture comprising a heparinized collagen matrix. For example, a schematic depiction of the preparation of affinity bound electrochemically aligned collagen suture is presented. See, FIG. 9. Following fabrication, ELAC sutures may undergo genipin crosslinking, peracetic acid ethanol treatment (Pet) and/or (3-dimethylaminopropyl)-3-ethylcarbodiimide-N-hydroxysuccinimide (EDC/NHS) conjugation of heparin before loading of a biologically active compound (e.g., PDGF-BB).

Following peracetic acid treatment alone, a reduction in tensile strength and modulus of collagen sutures was observed by 13% and 36%, respectively. However, when peracetic acid treatment is performed in combination with EDC/NHS heparin conjugation, this reduction in tensile strength and modulus was reversed such that a 34% and 76% increase in strength and Young modulus of the peracetic acid/heparinized treated collagen sutures, respectively, was observed. Therefore, the final strength of sutures following heparin conjugation was not significantly different from the baseline genipin crosslinked sutures. See, FIG. 10.

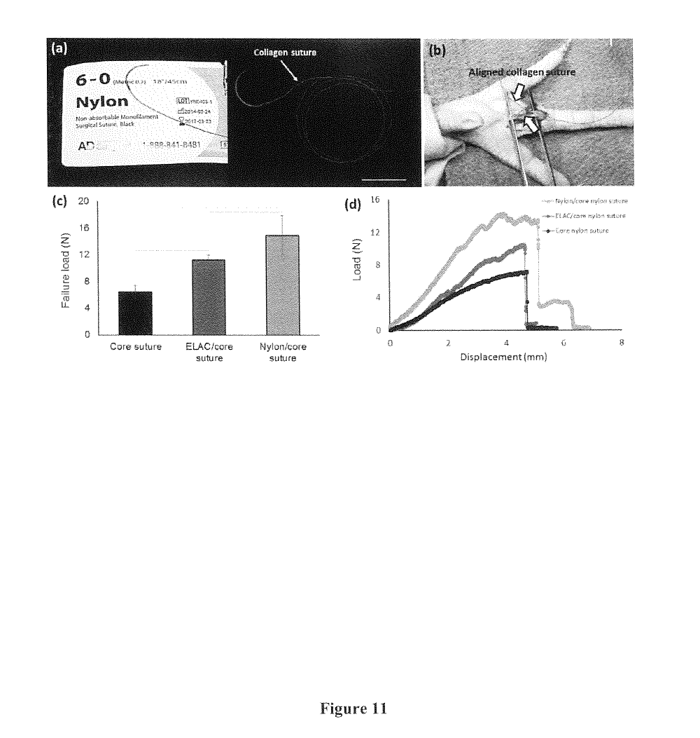

Aligned collagen sutures had similar dimensions as compared to standard 6-0 nylon sutures. See, FIG. 11A. To illustrate the suturability of a lacerated tendon with ELAC sutures and to demonstrate that the ELAC suture can augment the repair of a tendon, a lacerated flexor tendon in chicken claw was sutured with ELAC sutures. See, FIG. 11B. The core nylon suture alone provided a tensile failure load of 6.4±0.9 N. Augmentation of core nylon suture with ELAC or nylon epitendinous sutures improved the strength of the sutured tendon significantly by 75% and 130% (p<0.05), respectively. See, FIGS. 11C and 11D. Tendons sutured with an ELAC epitendinous suture had significantly lower failure load (11.2±0.7 N) than those sutured with 6-0 nylon epitendinous suture (14.9±2.9 N).

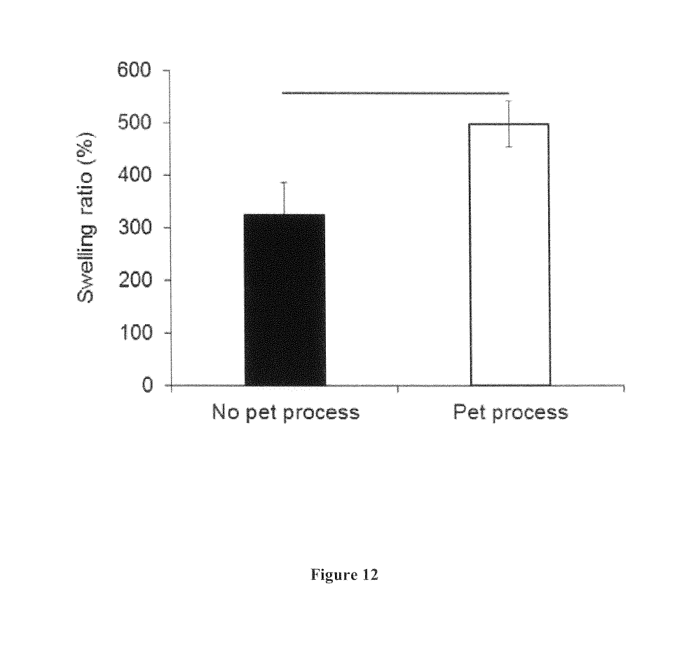

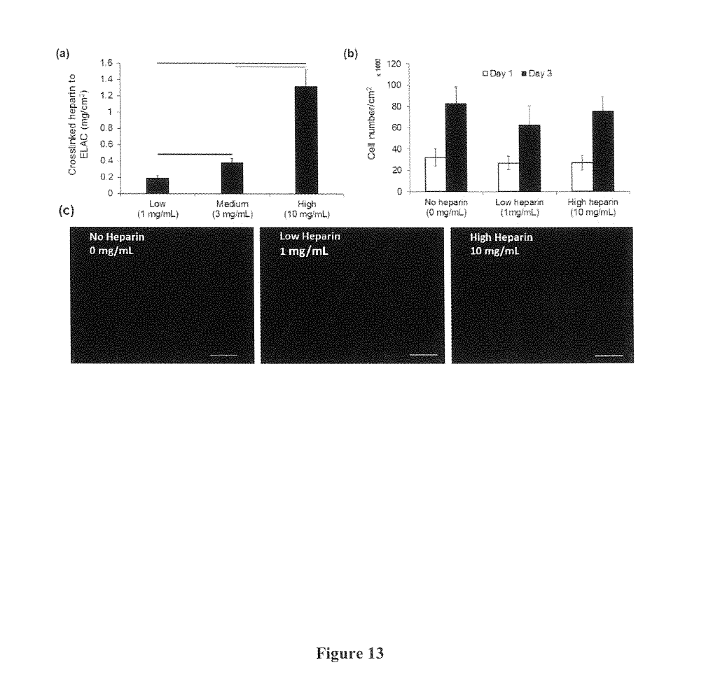

Following a Pet process as described herein, a swelling ratio of collagen sutures increased by 54% from 324 wt % to 498 wt %. See, FIG. 12. This process also bleached the dark blue color of the collagen sutures resulting from genipin crosslinking to a light brown tone. Increasing the concentration of heparin in the crosslinking solution from 1 mg/ml to 3 mg/ml and 10 mg/ml elevated the amounts of heparin which were crosslinked to collagen sutures from 0.19±0.02 mg/cm² to 0.38±0.05 and 1.32±0.2 mg/cm² of collagen suture surface. See, FIG. 13A. Results demonstrated that the cell proliferation on collagen sutures was not affected by the amount of conjugated heparin to collagen suture. See, FIGS. 13B and 13C, respectively.

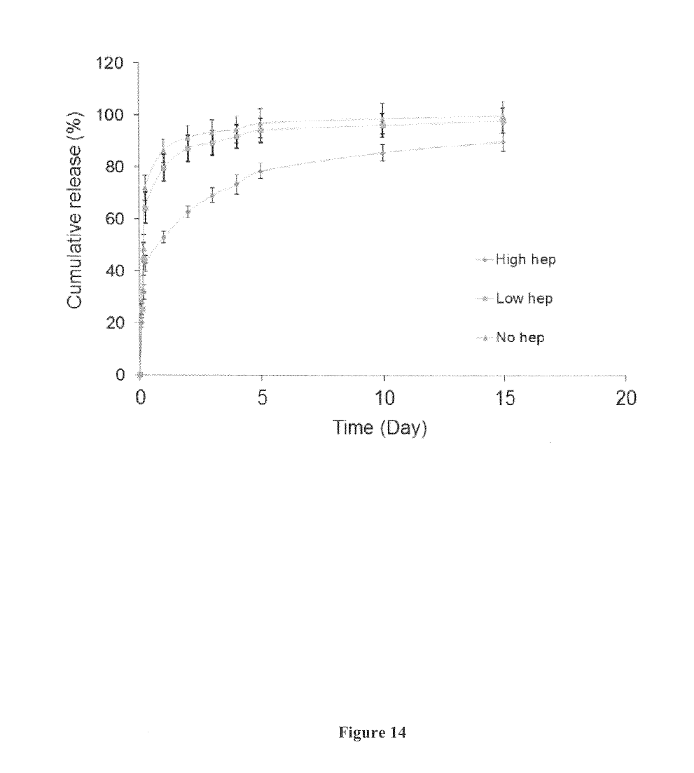

PDGF-BB release from 1 mg/ml heparin conjugation group was not different from that of the collagen sutures without heparin. ELAC sutures treated with a relatively high concentration heparin solution (10 mg/ml) attained a prolonged release over the 15 days period. PDGF-BB release by day 1 from collagen sutures without heparin and treated with low heparin were 86.3% and 79.6%, respectively. Collagen sutures treated with the highest concentration heparin released significantly less PDGF-BB at day 1 (52.9%). In the time span between day 2 to day 10, the collagen sutures conjugated with the highest amount of heparin solution released 32% of attached PDGF-BB and the sutures treated with low concentration of heparin solution released 16.6% of attached PDGF-BB. For the rest of the results, only the sutures conjugated with the highest dose of heparin is reported. See, FIG. 14.

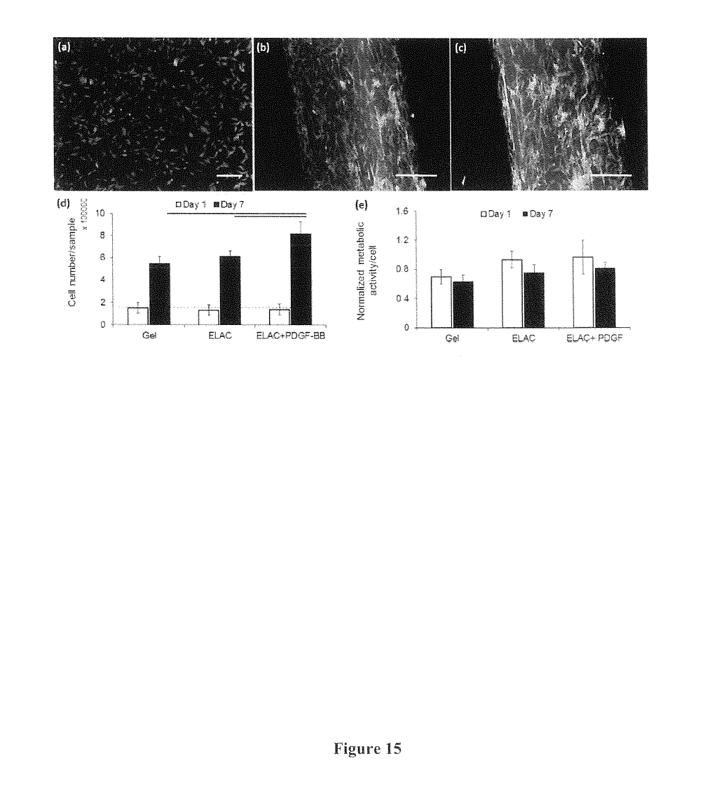

Fluorescent microscope imaging of the cells seeded on sutures had elongated morphologies along the long axis of the collagen suture. See, FIGS. 15A-C. Delivery of PDGF-BB increased the proliferation of cells significantly (p<0.05) and didn't have a significant effect on cell metabolic activities in all of the three groups. DNA quantification showed the cell content on collagen sutures with PDGF-BB were 37% and 71% greater than that on collagen sutures without the growth factor and collagen gel samples, respectively. See, FIGS. 15D-E.

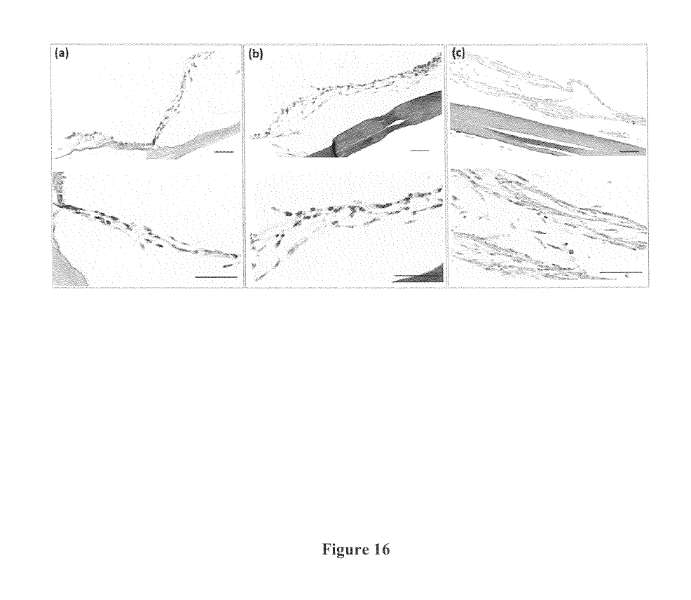

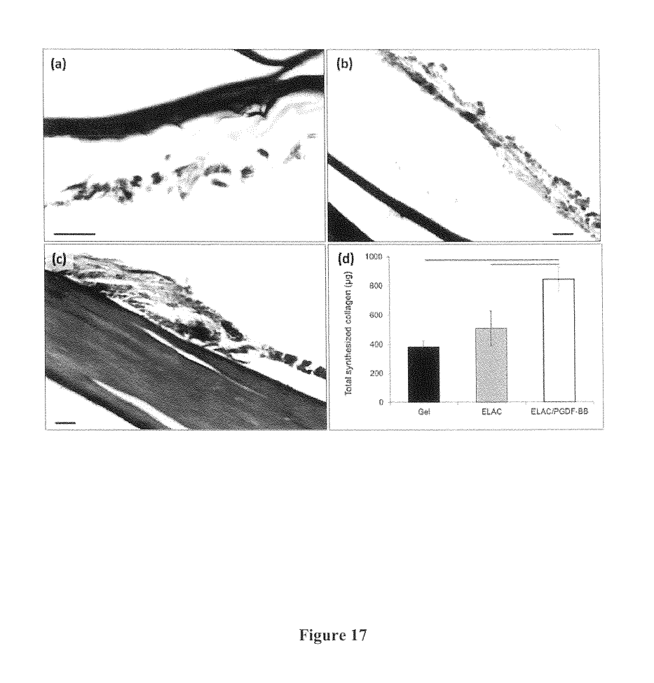

H&E staining of samples after 3 weeks of culture showed a significantly thicker layer of cells on the surface of a collagen suture with affinity bound PDGF-BB as compared to sutures without PDGF-BB and/or a collagen gel. See, FIGS. 16A-C. Masson's thrichrome stained sections demonstrated the presence of de novo collagen in the cells layers at day 21 time point with a more pronounced staining intensity for the growth factor treated group. Cell synthesized soluble collagen was quantified in the culture medium by sircol assay. See, FIGS. 17A-D.

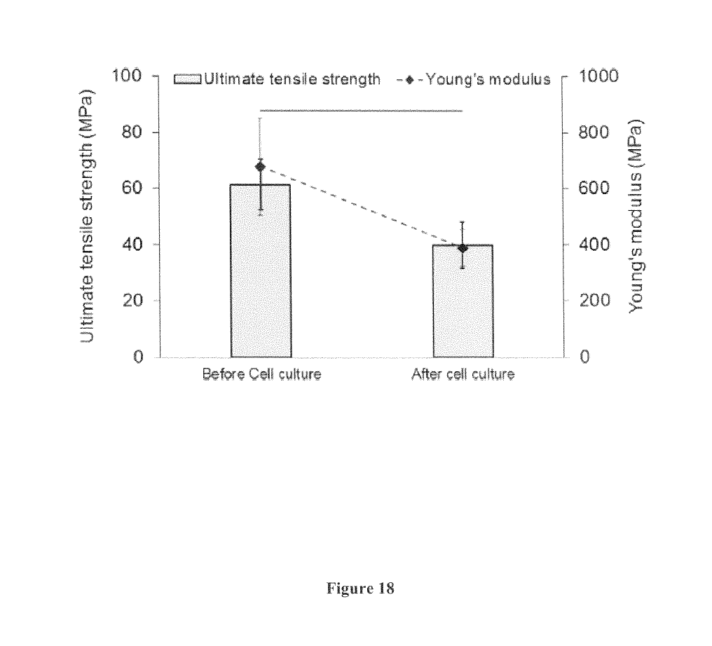

Amount of soluble collagen synthesized by cells on ELAC sutures conjugated PDGF-BB was 65% and 120% higher than ELAC sutures without growth factor and random collagen gel samples, respectively. Tensile strength and Young's modulus of sutures cultured with cells for 3 weeks showed a significant decrease of 34% and 42%, respectively. See, FIG. 18.

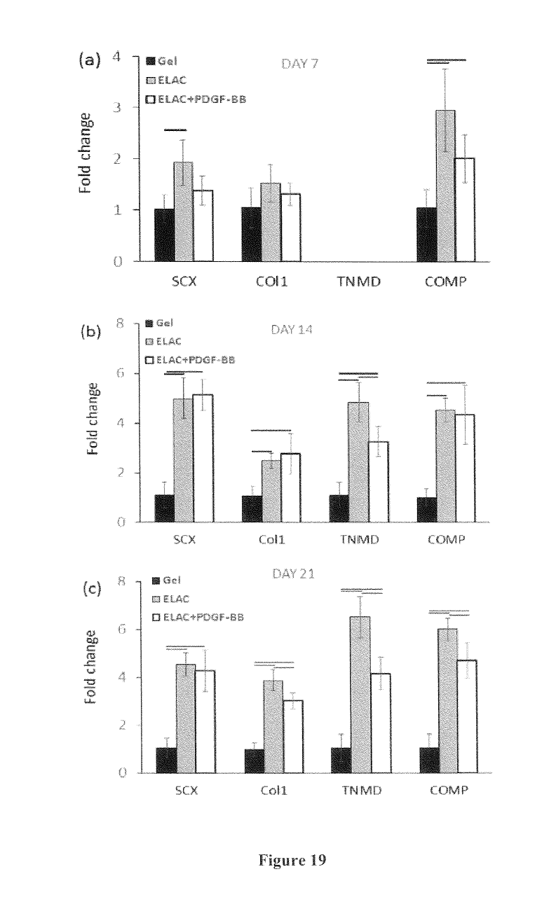

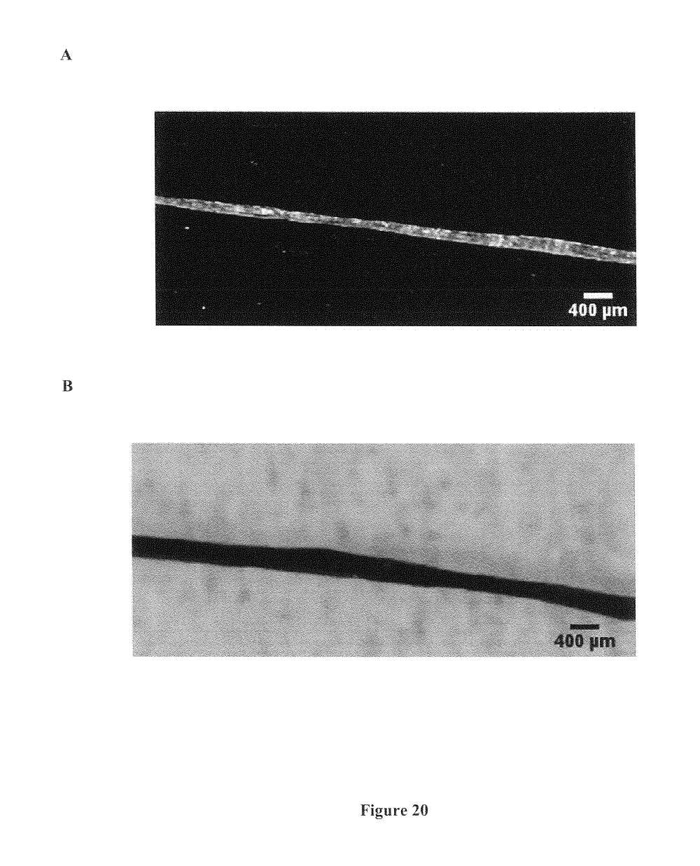

Real time-PCR analysis showed upregulation of Collagen I, SCX, COMP, and TNMD markers from day 7 up to day 21 on collagen sutures both with and without affinity bound PDGF-BB. See, FIG. 19. However, upregulation of most of these markers were reduced moderately following the conjugation of PDGF-BB at day 21 of culture (p<0.05). Tendon-specific markers of tenomodulin showed a late upregulation of 3.3±0.6 and 4.1±0.7 fold after 14 and 21 days on collagen sutures with PDGF-BB, and, 4.85±0.80 and 6.54±0.87 fold after 14 and 21 days on collagen sutures without PDGF-BB.

These results demonstrate that electrochemically aligned collagen sutures can be successfully functionalized with heparin to prolong PDGF-BB delivery. The conjugation process does not compromise the mechanical strength of threads. H&E staining showed the cell layer on aligned collagen sutures with affinity bound PDGF-BB and aligned collagen sutures without growth factor and random collagen gel after 3 weeks of cell culture. Higher magnification images of H&E stained samples showed the differences in cell layer thicknesses in all three groups.

Cell layer thickness on collagen sutures with affinity bound PDGF-BB is greater than cells on collagen suture without growth factor and also the collagen gel. Cell morphologies visualized by Alexa-fluor staining of cellular actin filaments demonstrated round and isotropic morphologies with no directional elongation on collagen gel as compared to those on collagen sutures without and/or with affinity bound PDGF-BB that had higher unidirectional elongation and anisotropic morphologies. The results of DNA quantification showed a higher proliferation rate for cells on collagen sutures with affinity bound PDGF-BB than cells on collagen sutures without growth factor and/or a collagen gel. The net effect of PDGF-BB on cells was such that the metabolic activity, cell proliferation, and synthesized collagen by cells were significantly increased. On the other hand, delivery of PDGF-BB had a moderately deleterious effect on tenogenic expression such that markers were more strongly expressed on ELAC threads than threads loaded with PDGF-BB. PDGF-BB has been reported to affect the differentiation of MSCs in various tendon regeneration applications.

It has been shown that PDGF-BB released from PLGA particles improved adipose-derived MSC proliferation and upregulation of tendon-related markers like tenomodulin, scleraxis, or tenascin. Cheng et al., “Platelet-derived growth-factor-releasing aligned collagen-nanoparticle fibers promote the proliferation and tenogenic differentiation of adipose-derived stem cells” Acta Biomater. 10:1360-1369 (2014). It has also been shown that PDGF-BB to promote tenogenic differentiation of the tendon stem cells to active tenocytes. Zhang et al., “Platelet-rich plasma releasate promotes differentiation of tendon stem cells into active tenocytes” Am. J. Sports Med. 38:2477-2486 (2010). PDGF-BB was also used to enhance the tendon healing process via enhancing the cell proliferation and matrix production.

Physical absorption of PDGF-BB by direct soaking of porous collagen scaffolds in growth factor laden solutions for delivery of PDGF-BB has been reported. Lynch et al., “Platelet-derived growth factor composition and methods for the treatment of tendon and ligament injuries” Biomimetic Therapeutics I, Editor, 2010. USA. While this method is a simple single step process, release of growth factor occurs over a matter of hours. For example, a fibrin/heparin scaffold delivery system designed for delivery of PDGF-BB which attained sustained delivery profile over 10 days. This design was a non-load bearing sheet which is inserted with the tendon-proper. Placement of the sheet was accompanied by the creation of a gap invasively between the two ends of the tendon. Sutures provide a more practical and less invasive mean to deliver the growth factor.

In this vein, sutures may be coated with growth factor doped gelatin by simple dipping. While this method directly transfers the growth factor to place of injury, separation of the gelatin coating while threading the suture is a possibility. Furthermore, fast degradation of gelatin coating and lack of chemical bonding between growth factor and gelatin is likely to shorten the release profile timeline to less than a few days. In the embodiments described herein, the present invention contemplates a method of suture administration having several advantages. In one embodiment, the suture comprises aligned collagen threads that provide a tenoinductive effect.

Previous studies have shown such tenoinductive effect on human mesenchymal stem cells via the biomimetic topographical features presented by ELAC sutures (data not shown). The data presented herein demonstrates similar effects on tendon-derived cells from chickens, confirming a topographical tenoinductive effect of ELAC suture that is efficacious across species and cell types. For example, a tendon-derived cell population can be heterogeneous comprising, for example, tenocytes, tenoblasts and/or MSCs. Therefore, ELAC sutures as described herein may not only be providing a tenogenic differentiation in the context of MSCs, but also may help maintain a phenotype of harvested tenocytes and tenoblasts as demonstrated by upregulation of tendon-specific and tendon-related markers as compared to random collagen gel which lacks alignment and compaction.

It is well known that PDGF-BB promotes proliferation of tendon derived cells as well as MSCs. Lepisto et al., “Effects of homodimeric isoforms of platelet-derived growth factor (PDGF-AA and PDGFBB) on wound healing in rat” J. Surg. Res. 53:596-601 (1992); and Wroblewski et al., “PDGF BB stimulates proliferation and differentiation in cultured chondrocytes from rat rib growth plate” Cell Biol. Int. Rep. 16:133-144 (1992). A positive effect of PDGF-BB on matrix production has been demonstrated through an increase in the TGFβ-1 expression. Pierce et al., “Platelet-derived growth factor and transforming growth factorbeta enhance tissue repair activities by unique mechanisms” J. Cell Biol. 109:429-440 (1989). However, in vivo results illustrated that there is no paired effect between PDGF-BB and TGFβ-1. Hildebrand et al., “The effects of platelet-derived growth factor-BB on healing of the rabbit medial collateral ligament. An in vivo study” Am. J. Sports Med. 26:549-554 (1998).

Earlier studies demonstrated that a single dose delivery of a growth factor does not effect repair processes because of the fast clearance of the growth factor from the injury site. Robinson et al., “Sustained release of growth factors, In vivo” 16:535-540 (2002). As shown herein, heparinized collagen sutures prolong PDGF-BB release and/or delivery over a course of at least fifteen (15) days which resulted in a significant improvement in the proliferation of tendon derived cells. As it is generally believed that a tendon fibroblast can appear at an injury site within 2-5 days, a growth factor release and/or delivery of approximately ten (10) days provides improved healing outcomes in vivo.

Histology results confirmed that release of PDGF-BB from a collagen suture enhances cell proliferation as demonstrated by a thicker cell layer on collagen sutures with affinity bound PDGF-BB. Collagen production by cells as quantified by the amount of soluble collagen into the medium was highest for the PDGF-BB treated group. In support of this result, cell-deposited collagen, as measured in Masson's trichrome stained images, was most intense for the growth factor treated group. Although it is not necessary to understand the mechanism of an invention, it is believed that increased collagen amounts detected in cell culture medium of the PDGF-BB delivery group was an outcome of the greater number of cells instead of greater degree of collagen production by each cell.

In one embodiment, the present invention contemplates a collagen suture as a drug delivery platform. It was observed that, unmodified collagen sutures lose mechanical properties by about 34% in the cell culture after 3 weeks, likely due to a combination of cellular and physical degradation. These results suggest that modified (e.g., load bearing) synthetic sutures are capable of providing long term mechanical stability, thereby allowing complete tissue healing to occur. In one embodiment, the modified, load bearing, sutures can become biologic carriers (e.g., for example, PDGF-BB) to accelerate tissue healing.

As demonstrated herein, there is an upregulation of tendon related markers (e.g., collagen I, tenomodulin, scleraxis, COMP) on aligned collagen sutures with and without the PDGF-BB. However, at the day 14 and 21 time points, collagen I and tenomodulin gene expression were moderately lower for sutures comprising PDGF-BB as opposed to sutures without PDGF-BB. Although it is not necessary to understand the mechanism of an invention, it is believed that electrochemically aligned collagen sutures guide MSCs toward a tenogenic lineage via topographical cues and in the absence of growth factors.

Similarly, addition of BMP-12 (GDF-5) reduces the expression of scleraxis and tenomodulin moderately by day 14 (data not shown). Despite a moderate reduction in tendon-specific and tendon-related gene expression of cells in the presence of PDGF-BB, the amount of synthesized collagen in culture medium was enhanced. Although it is not necessary to understand the mechanism of an invention it is believed that a reduction in the differentiation under PDGF-BB delivery can be compensated by the beneficial effects of PDGF-BB on cell proliferation.

Generally, it is well appreciated that proliferation and differentiation cues oppose each other and a successful repair outcome depends on balanced attainment of the both. While the present results indicate a potential for enhancement in tendon repair via heparinized collagen threads, a formulation can be optimized further in terms of the level of heparinization and the amount of growth factor conjugated to collagen suture to achieve the best outcome in terms of cell proliferation and tenogenic markers expression. In doing so, delivery of PDGF-BB can be increased in the first week to increase cell number by proliferation. Therefore, prolonging the PDGF-delivery to longer time points may not be ideal by curbing the latent differentiation under the topographical tenogenic cues provided by aligned collagen fibers.

IV. Crosslinker Strength Comparison: Genipin Versus EDC/NHS

In one embodiment, the present invention contemplates a method for producing and crosslinking a plurality of robust collagen threads through electrocompaction to create a crosslinked ELAC collagen suture. In one embodiment, the crosslinker comprises genipin, a naturally derived crosslinker. Although it is not necessary to understand the mechanism of an invention it is believed that the crosslinking increases the tensile strength of these electrocompacted collagen threads.

Investigation of the appropriateness of genipin crosslinked collagen sutures for tendon repair showed that such sutures were mechanically robust, with properties converging to the level of native tendon, in addition to supporting cellularization in vitro. Younesi et al., “Tenogenic Induction of Human MSCs by Anisotropically Aligned Collagen Biotextiles,” Adv. Funct. Mater., vol. 24, no. 36, pp. 5762-5770, 2014. The tunable nature of scaffolds for other applications, such as cartilage tissue engineering, has also been demonstrated and implies that such scaffolds might be tailored into a suitable suture material. Younesi et al., “A Biomimetic collagen template for mesenchymal condensation based regeneration” Acta Biomater., vol. 30, pp. 212-221, 2015. In one embodiment, the crosslinking comprises 1-ethyl-3-(3-dimethylaminopropyl)-carbodiimide-N-hydroxy-succinimide (EDC/NHS). In one embodiment, EDC/NHS crosslinking produces a plurality of collagen threads with suitable mechanical properties for clinical use.

Although it is not necessary to understand the mechanism of an invention, it is believed that the mechanical properties, strength and stiffness in particular, of collagen and other naturally derived biomaterials have been shown to decrease with enzymatic degradation and remodeling in vitro as well as in vivo. Afua Annor et al., “Effect of enzymatic degradation on the mechanical properties of biological scaffold materials,” Surg. Endosc., vol. 26, no. 10, pp. 2277-5, 2012. Because of this, it is expected that the mechanical properties may decrease over time and that significant early incompatibilities in mechanical properties can be reflected in measures of biocompatibility. Accordingly, EDC/NHS crosslinked collagen threads might be expected to prevent the naturally occuring decrease in unmodified collagen threads to provide an ideal long term implantable material.

Collagen crosslinking procedures using EDC/NHS has aesthetic advantages to genipin crosslinking for several reasons. First, and in contrast to genipin, FDA-approved collagen based medical devices crosslinked using EDC and NHS are known (Collamend by Bard, acellular, EDC/NHS crosslinked porcine dermis). Second, at low molar concentrations EDC/NHS crosslinking does not change color or undergo a significant change in porosity of collagen devices as has been reported for genipin crosslinked collagen devices. Vrana et al., “EDC/NHS crosslinked collagen foams as scaffolds for artificial corneal stroma,” J. Biomater. Sci. Polym. Ed., vol. 18, no. 12, pp. 1527-45, 2007. Third, crosslinking using EDC and NHS takes only a few hours at most at room temperature while genipin crosslinking requires three days minimum at 37° C. Uquillas et al., “Genipin crosslinking elevates the strength of electrochemically aligned collagen to the level of tendons,” J. Mech. Behav. Biomed. Mater., vol. 15, pp. 176-189, 2012; and Yang C., “Enhanced physicochemical properties of collagen by using EDC/NHS-crosslinking,” Bull. Mater. Sci., vol. 35, no. 5, pp. 913-918, 2012. Overall, EDC/NHS is an FDA approved modification have a faster crosslinking regime which does not affect the aesthetic appearance or structure of devices. Even so, the data below suggests that genipin has superior mechanical properties over EDC/NHS thereby making genipin a preferred crosslinker for clinically used ELAC collagen medical devices.

Before crosslinking, ELAC threads were soaked in 1×PBS for 1 hour at 37° C. in order to promote fibrillogenesis and increase the baseline strength of threads. Uquillas et al., “Effect of phosphate buffered saline concentration and incubation time on the mechanical and structural properteis of electrochemically aligned collagen threads,” Biomed. Mater., vol. 6, no. 3, 2011. Threads were then crosslinked in MES buffer (2-(N-morpholino)ethanesulfonic acid, Sigma, Missouri) using EDC/NHS (Fisher Scientific, Waltham, Mass.) using molar ratios of 1:10:25, 1:25:50, and 1:100:250 (collagen:EDC:NHS, or, low, medium, and high molar ratios respectively) for two hours. After two hours the crosslinking solution was refreshed and the reaction was allowed to continue overnight. After crosslinking was complete, threads were rinsed in 0.1 M sodium phosphate for 30 minutes and then rinsed with 1×PBS three times for 15 minutes each time.

The mechanical properties of threads crosslinked at the different molar ratios were then measured in accordance with Example XIII. FIG. 20A. ELAC threads were also crosslinked using genipin and tested. Threads were treated with 1×PBS as previously mentioned and then transferred directly to a 2% (weight/volume) solution of genipin in 90% ethanol. FIG. 20B.

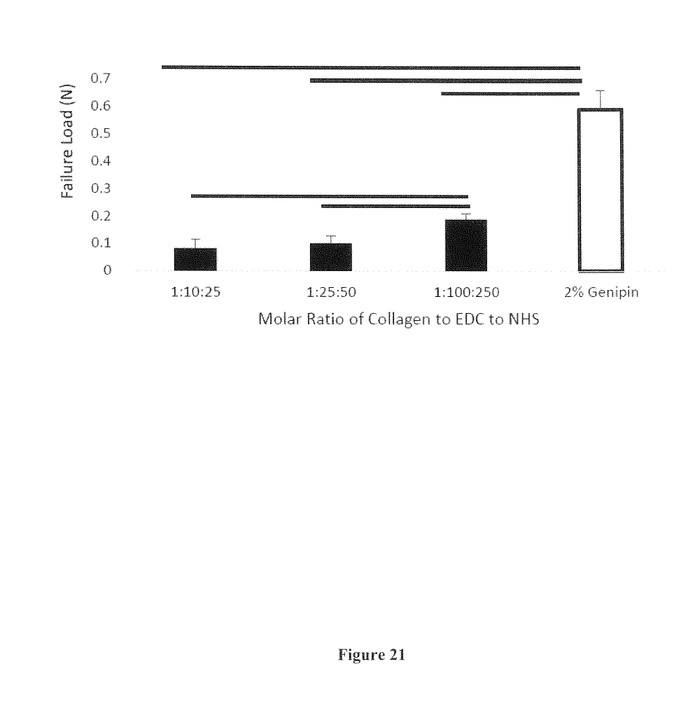

Crosslinking was carried out for 3 days at 37° C., after which threads were rinsed in 1×PBS and tested. The highest molar ratio (1:100:250) of EDC/NHS used had significantly greater load to failure than the medium (p=0.02) and low molar EDC/NHS ratios (p=0.012) but was still significantly lower than the load to failure for genipin crosslinked ELAC threads. Table 4, and FIG. 21.

TABLE 4
Failure loads for low, medium and high molar ratios for
EDC/NHS crosslinking and p-values for Mann-Whitney tests
comparing failure loads to those of 2% genipin crosslinked
threads

| | | | P-value from |
|---|---|---|---|
| | | | Mann-Whitney |
| | Failure | | comparison |
| Crosslinking Molar Ratio | Load | Standard | to Genipin |
| (Collagen:EDC:NHS) | (N) | Deviation | (α = 0.05) |
| 1:10:25 | 0.083 | 0.032 | 0.002 |
| 1:25:50 | 0.102 | 0.026 | 0.002 |
| 1:100:250 | 0.189 | 0.021 | 0.002 |
| 2% Genipin | 0.59 | 0.07 | — |

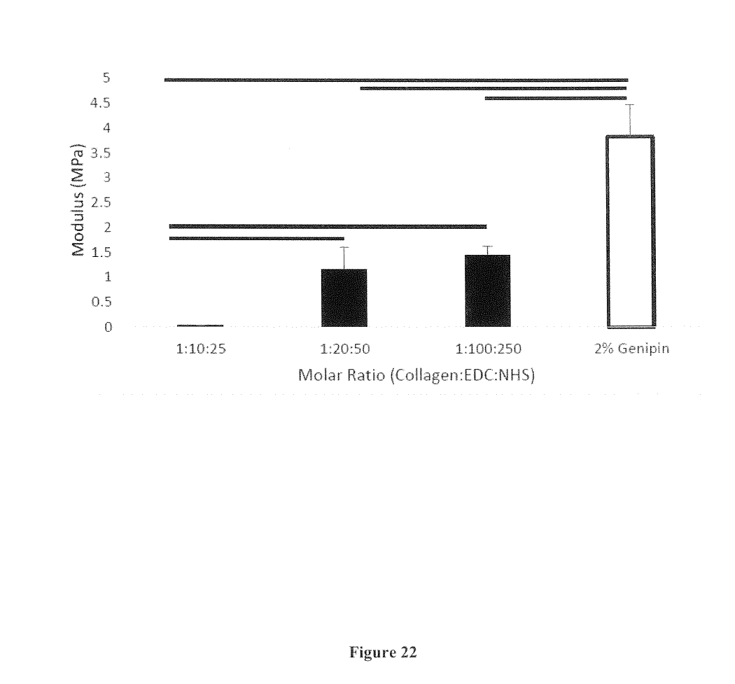

The modulus values of threads increased significantly with increase in EDC/NHS molar ratio from low to medium (p=0.002) but not from medium to high (p=0.792). The moduli of threads were significantly less than threads crosslinked in 2% genipin for all molar ratios (low, p=0.002; medium, p=0.003; high, p=0.002). FIG. 22.

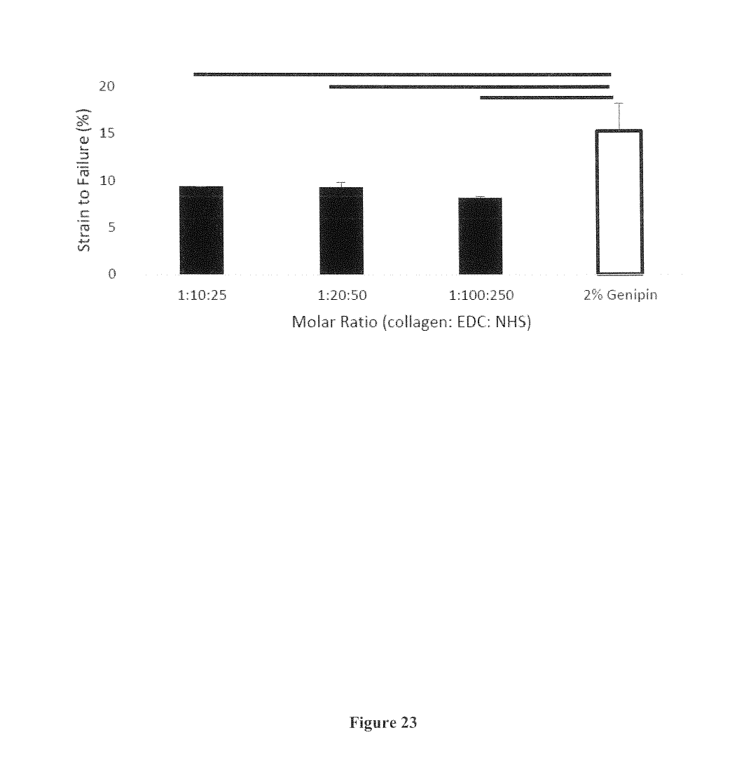

There was no significant difference in strain to failure among different EDC/NHS molar ratios of crosslinking, but again, all were significantly lower than the strain to failure of threads crosslinked using 2% genipin (low, p=0.002; medium, p=0.003; high, p=0.002). FIG. 23.

The corresponding increase in molar ratio with increasing load to failure, modulus, and decreasing strain to failure for these experiments indicate that the additional EDC and NHS were consumed in additional crosslinking reactions that increased the strength and stiffness of threads. However, threads were still significantly weaker and less stiff than 2% genipin crosslinked threads. As studies rarely report using a molar ratio as high as 1:100:250, and some report adverse effects at higher molar ratios, it was decided that pursuing an alternative solvent was the next step to increase mechanical strength to the level of 2% genipin crosslinked threads rather than increasing the molar ratio further. Gratzer et al., “Control of pH alters the type of cross-linking produced by 1-ethyl-3-(3-dimethylaminopropyl)-carbodiimide (EDC) treatment of acellular matrix vascular grafts,” J. Biomed. Mater. Res., vol. 58, no. 2, pp. 172-179, 2001; and Shepherd et al., “The process of EDC-NHS cross-linking of reconstituted collagen fibres increases collagen fibrillar order and alignment,” APL Mater., vol. 3, no. 1, pp. 1-7, 2015.

As the strength of ELAC threads crosslinked using EDC/NHS in MES buffer did not approach the preferred level of genipin crosslinked threads, the crosslinking solution was changed. EDC/NHS crosslinking is most efficient in slightly acidic (pH 4.5) conditions and in the absence of extra carboxyl and amine groups. However, phosphate buffers and neutral solutions are acceptable environments for crosslinking although efficiency of the reaction may decrease. Crosslinking collagen using EDC/NHS has been reported to employ alcohols or organic solutions and to have success in terms of stability and strength of collagen devices. Barnes et al., “Cross-linking electrospun type II collagen tissue engineering scaffolds with carbodiimide in ethanol.,” Tissue Eng., vol. 13, no. 7, pp. 1593-605, 2007. As such, ELAC threads were crosslinked using EDC/NHS at the molar ratio of 1:100:250 in varying percentages of ethanol, 50-100%, with either water or MES as the co-solvent.

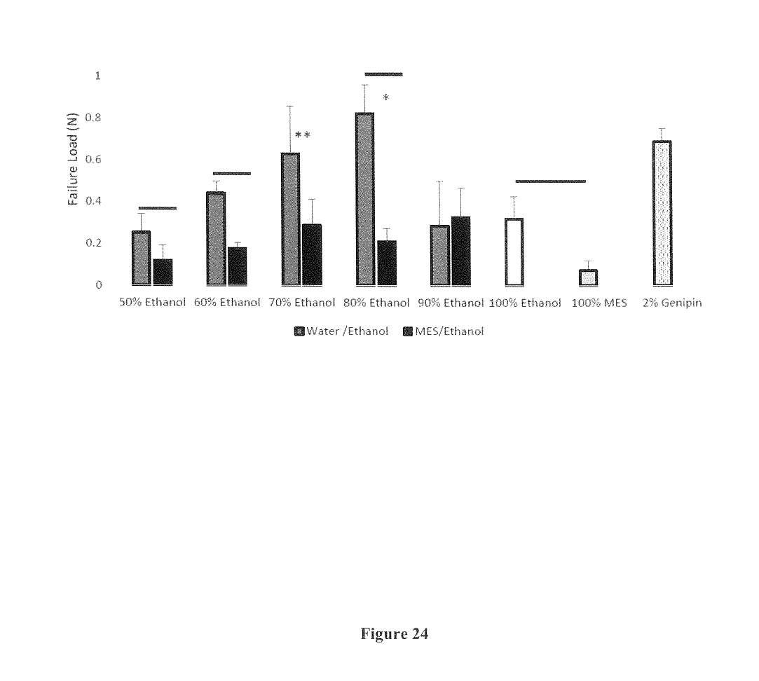

Threads crosslinked in a solution of water and ethanol had significantly greater failure loads than threads crosslinked in a solution of MES buffer and ethanol, except at a concentrations of 70% and 90% ethanol. Table 5, and FIG. 24.

TABLE 5
Mean failure loads for ELAC threads crosslinked in variable
percentages of ethanol and water and p values for Mann
Whitney tests comparing failure loads those of 2% genipin
and comparable MES/ethanol co-solvent percentages

| | | | P-value for |
|---|---|---|---|
| | | P-value | comparison to |
| | | for comparison | comparable |
| % of Ethanol | Mean Failure | to 2% genipin | MES co-solvents |
| in Water | Load (N) | (α = 0.05) | (α = 0.05) |
| 50 | 0.292 | 0.002 | 0.036 |
| 60 | 0.428 | 0.01 | 0.012 |
| 70 | 0.53 | 0.94 | 0.095 |
| 80 | 0.8 | 0.002 | 0.012 |
| 90 | 0.28 | 0.006 | 1 |
| 100% | 0.32 | 0.006 | 0.012 |
| 2% Genipin | 0.59 | — | — |

All threads except those crosslinked in 70% and 80% ethanol/water had significantly lower loads to failure than threads crosslinked in 2% genipin. Failure loads for threads crosslinked in 70% ethanol/water were not significantly different from failure loads of threads crosslinked in 2% genipin. Failure loads of threads crosslinked in 80% ethanol were significantly greater than failure loads of threads crosslinked in 2% genipin. Tables 5 and Table 6; and FIG. 24.

TABLE 6
Mean failure loads for ELAC threads crosslinked in variable
percentages of ethanol and MES and p values for Mann Whitney
tests comparing failure loads to those of 2% genipin

| | | P-value of Mann-Whitney |
|---|---|---|
| % of Ethanol in | | Test for comparison to |
| MES Buffer | Mean Failure Load (N) | Genipin (α = 0.05) |
| 50 | 0.118 | 0.002 |
| 60 | 0.181 | 0.002 |
| 70 | 0.32 | 0.002 |
| 80 | 0.22 | 0.002 |
| 90 | 0.24 | 0.002 |
| 100% | 0.3 | 0.003 |
| 2% Genipin | 0.59 | — |
| 100% MES | 0.189 | 0.004 |

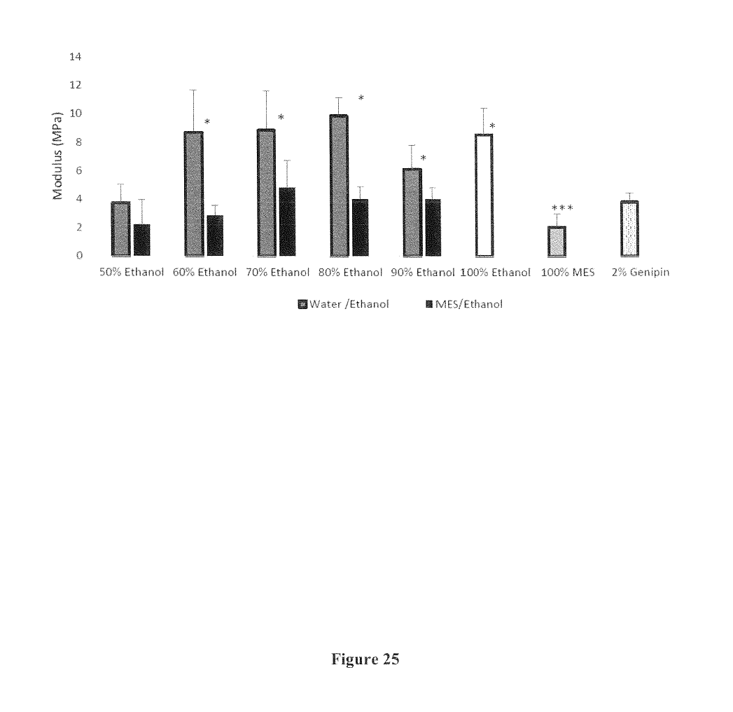

Modulus for threads crosslinked in 50% ethanol were not significantly different from modulus values for threads crosslinked in 2% genipin. Modulus values for all other threads crosslinked in ethanol and water were significantly greater than those for threads crosslinked in 2% genipin. Modulus values for threads crosslinked in MES buffer and ethanol were not significantly different than those for threads crosslinked in 2% genipin. Threads crosslinked in pure MES buffer had significantly lower modulus values than 2% genipin crosslinked threads. Table 7 and Table 8; and FIG. 25.

TABLE 7
Modulus values of ELAC threads crosslinked in ethanol and
water and the p-values of Mann Whitney tests comparing those
values to modulus values for ELAC threads crosslinked in
2% genipin

| | | p-value | Greater or less than |
|---|---|---|---|
| | Modulus | compared to | 2% genipin (α = |
| % Ethanol in Water | (MPa) | 2% genipin | 0.05) |
| 50% Ethanol | 3.76 | 0.526 | — |
| 60% Ethanol | 8.69 | 0.002 | greater |
| 70% Ethanol | 8.86 | 0.006 | greater |
| 80% Ethanol | 9.88 | 0.002 | greater |
| 90% Ethanol | 6.117 | 0.038 | greater |
| 100% Ethanol | 8.55 | 0.034 | greater |
| 2% Genipin | 3.82 | — | — |

TABLE 8
Modulus values of ELAC threads crosslinked in ethanol and MES
buffer and the p-values of Mann Whitney tests comparing those
values to modulus values for ELAC threads crosslinked in
2% genipin.

| | | p-value | Greater or less |
|---|---|---|---|
| % Ethanol in MES | Modululus | compared to | than 2% genipin |
| Buffer | (MPa) | 2% genipin | (α = 0.05) |
| 50% Ethanol | 2.23 | 0.3 | — |
| 60% Ethanol | 2.88 | 0.124 | — |
| 70% Ethanol | 4.84 | 0.143 | — |
| 80% Ethanol | 4.03 | 0.943 | — |
| 90% Ethanol | 4.02 | 0.626 | — |
| 100% Ethanol | 8.55 | 0.034 | greater |
| 2% Genipin | 3.82 | — | — |
| 100% MES | 2.06 | 0.006 | less |

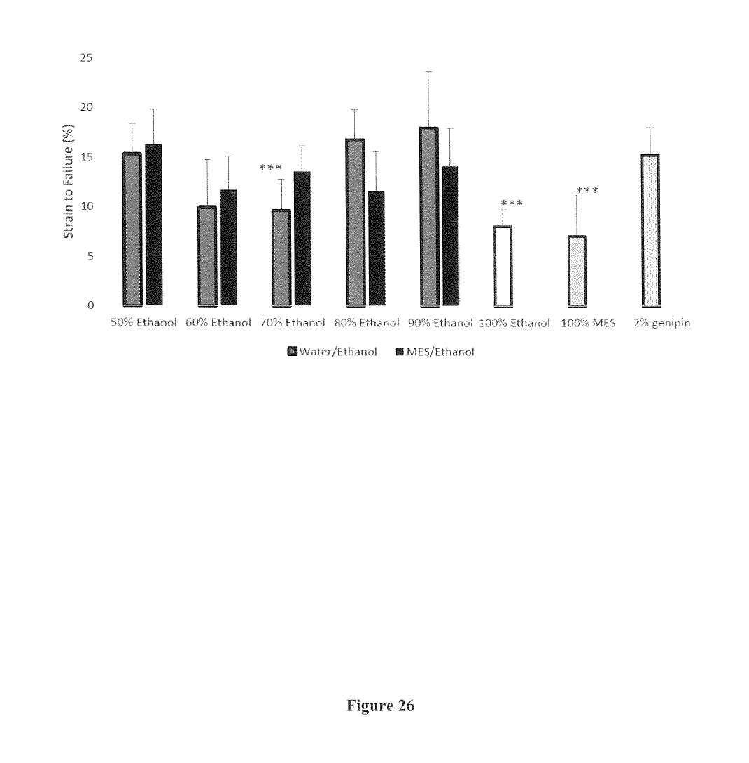

ELAC threads crosslinked in 70% ethanol/water, 100% ethanol and 100% MES buffer had significantly lower strain to failure than ELAC threads crosslinked in 2% genipin. Strain to failure for ELAC threads crosslinked in all other percentages of ethanol/water and ethanol/MES did not have significantly different strain to failure than ELAC threads crosslinked in 2% genipin. Table 9 and Table 10; and FIG. 26.

TABLE 9
Strain to failure of ELAC threads crosslinked in variables
percentages of ethanol and water and p-values of Mann-
Whitney tests comparing strain to failure to that of ELAC
threads crosslinked in 2% genipin

| % of Ethanol in | Strain to Failure | p-value compared to 2% |
|---|---|---|
| Water | (%) | genipin (α = 0.05) |
| 50% Ethanol | 15.34 | 0.508 |
| 60% Ethanol | 9.96 | 0.1 |
| 70% Ethanol | 9.62 | 0.256 |
| 80% Ethanol | 16.79 | 0.104 |
| 90% Ethanol | 18.02 | 0.417 |
| 100% Ethanol | 8.06 | 0.006 |
| 2% genipin | 15.25 | — |
| 100% MES | 6.99 | 0.015 |

TABLE 10
Strain to failure of ELAC threads crosslinked in variables
percentages of ethanol and MES buffer and p-values of
Mann-Whitney tests comparing strain to failure to that
of ELAC threads crosslinked in 2% genipin.

| % of Ethanol in | Strain to Failure | p-value compared to 2% |
|---|---|---|
| MES | (%) | genipin (α = 0.05) |
| 50% Ethanol | 16.3 | 0.609 |
| 60% Ethanol | 11.75 | 0.097 |
| 70% Ethanol | 13.62 | 0.011 |
| 80% Ethanol | 11.63 | 0.201 |
| 90% Ethanol | 14.14 | 0.521 |
| 100% Ethanol | 8.058 | 0.006 |
| 2% genipin | 15.25 | — |

The results of co-solvent testing show that ELAC threads crosslinked in 80% ethanol and water have significantly greater load to failure and modulus values than ELAC threads crosslinked in 2% genipin, as well as comparable strain to failure values to ELAC threads crosslinked in 2% genipin.

EDC/NHS crosslinking in 80% alcohol/aqueous solutions has been shown to produce optimal results. Vashist S., “Comparison of 1-Ethyl-3-(3-Dimethylaminopropyl) Carbodiimide Based Strategies to Crosslink Antibodies on Amine-Functionalized Platforms for Immunodiagnostic Applications,” Diagnostics, vol. 2, no. 3, pp. 23-33, 2012. While the exact reasons are unknown, it is possible that the specific percentage of alcohol and aqueous solution interact with collagen molecules such that molecules are an ideal distance for efficient EDC/NHS crosslinking. As such, significantly lower failure loads for threads crosslinked using ethanol/MES may be the result of ions in the buffer affecting repulsion between collagen molecules such that the efficiency of crosslinking is decreased. The increased modulus of EDC/NHS crosslinked ELAC threads compared to 2% genipin crosslinked ELAC threads is the result of the distance the respective crosslinking molecules are capable of bridging. As genipin molecules are capable of bridging larger distances, the extensibility of threads before failure may be greater. Furthermore, the higher modulus values for EDC/NHS crosslinked ELAC threads should not be a concern in terms of creating a stiffer thread material than an ELAC thread crosslinked in 2% genipin as the stiffness of both crosslinked and uncrosslinked collagen have been seen to rapidly decrease in vivo over time. Of interest to note is the increase in strain to failure for ELAC threads crosslinked in higher percentages of ethanol and water. Typically materials which increase in strength and stiffness show a decrease in strain to failure. In this case, the changing distance between crosslinking groups with changing water content in solution may allow for greater extension before failure. Crosslinking in 80% ethanol in water produces ELAC threads with comparable strength to those crosslinked in 2% genipin.

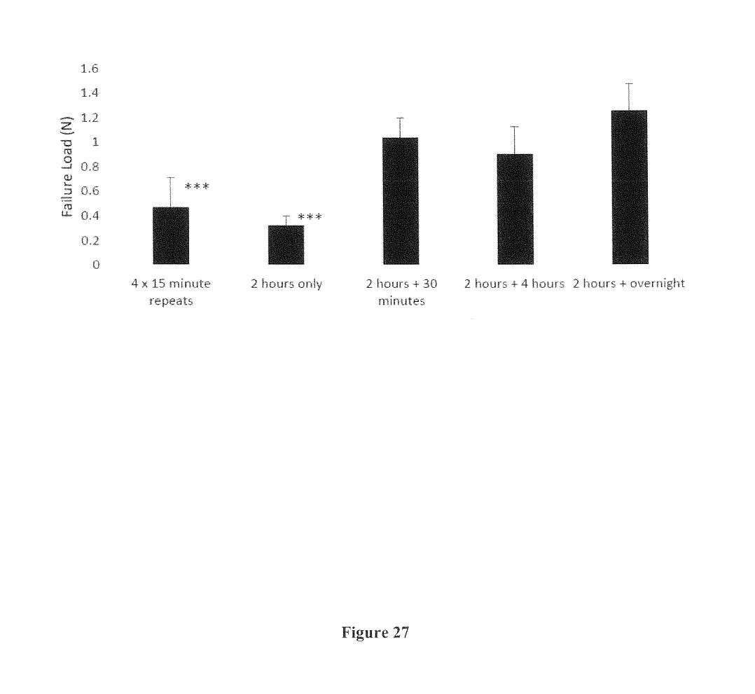

Initially, the crosslinking time for ELAC threads was extended beyond typical times for crosslinking—between two and four hours—noted in literature as the collagen molecules within ELAC threads are densely compacted. EDC/NHS crosslinking procedures were also tested at various times of incubation to assess it effects on the resultant mechanical properties of threads. ELAC threads were crosslinked using an EDC?NHS molar ratio of 1:100:250 in 80% ethanol for two hours and then crosslinked for variable lengths of time afterwards. One group of threads were crosslinked for two hours only, and one group was crosslinked four times for fifteen minutes each time. Results of mechanical testing showed that crosslinking for two hours alone resulted in a significantly lower failure load than the original regimen of two hours and then overnight. Table 11; and FIG. 27.

TABLE 11
Failure loads for threads crosslinked for different durations and
repetitions and the p-values for Mann-Whitney tests comparing
failure loads to those of ELAC threads crosslinked for two hours
and then overnight.

| Crosslinking Timing/ | Load to Failure | p-value for comparison to 2 |
|---|---|---|
| Repetitions | (N) | hours + overnight (α = 0.05) |
| 4 × 15 minute repeats | 0.472 | 0.035 |
| 2 hours only | 0.325 | 0.006 |
| 2 hours + 30 minutes | 1.04 | 0.52 |
| 2 hours + 4 hours | 0.908 | 0.023 |
| 2 hours + overnight in | 1.26 | — |
| 80% ethanol | | |

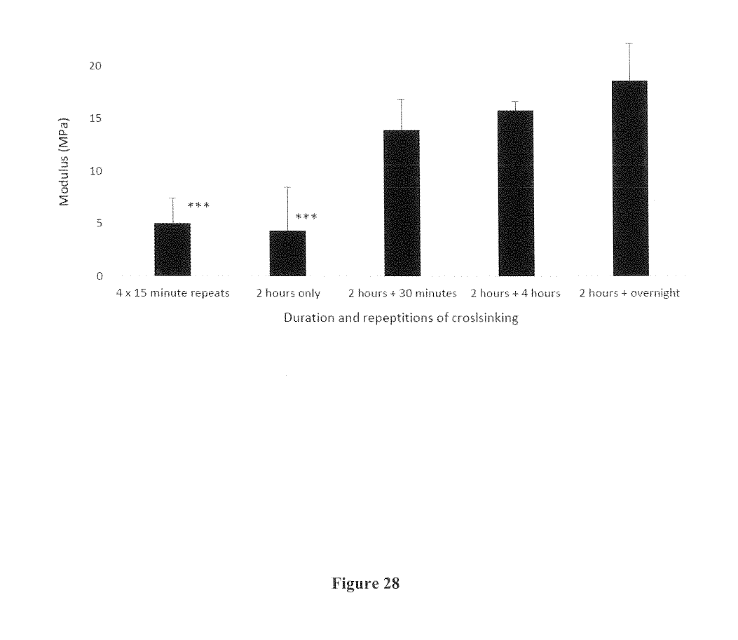

Results also showed that duration of crosslinking rather than the repetition of crosslinking resulted in failure load and modulus values closer to the original regimen of two hours of crosslinking followed by crosslinking overnight; Table 11 and Table 12; FIG. 27 and FIG. 28.

TABLE 12
Modulus values for threads crosslinked for different durations and
repetitions and the p-values for Mann-Whitney tests comparing
modulus values to those of ELAC threads crosslinked for two
hours and then overnight.

| Crosslinking Timing/ | | p-value for comparison to 2 |
|---|---|---|
| Repetitions | Modulus | hours + overnight (α = 0.05) |
| 4 × 15 minute repeats | 5.05 | 0.009 |
| 2 hours only | 4.35 | 0.006 |
| 2 hours + 30 minutes | 13.98 | 0.148 |
| 2 hours + 4 hours | 15.837 | 0.201 |
| 2 hours + overnight | 18.66 | — |

In particular, ELAC threads that were crosslinked for two hours and then for thirty minutes did not have significantly different failure loads modulus values when compared to ELAC threads crosslinked for two hours and then overnight. There were no significant differences in strain to failure among the different crosslinking regimens. FIG. 27. The increase in strength and stiffness with increasing repetitions and duration of these repetitions indicate that EDC/NHS crosslinking of ELAC threads may take more time given the density of collagen molecules packed into threads. The results of these experiments also demonstrate that the strength and stiffness of ELAC threads can be readily adjusted by adjusting the parameters of EDC/NHS crosslinking.

Overall, the data show that genipin crosslinked ELAC sutures are superior to those crosslinked with EDC/NHS unless the EDC/NHS is reacted in the presence of relatively high ethanol conentrations results.

### EXPERIMENTAL

### Example I

Preparation of an Antibacterial Collagen-Heparin Coating

Electrochemically aligned collagen (ELAC) sheets were synthesized as previously described (Uquillas and Akkus 2012). Briefly, acid-soluble monomeric collagen solution from bovine hide (Nutragen 6.4 mg/mL; Advanced Biomatrix, Tucson, Ariz.) was dialyzed free of salts. The dialyzed collagen solution was loaded between two planar carbon electrodes and an electric voltage of 30 V was applied. In the presence of an electric field, collagen monomers align along the isoelectric point and form a highly oriented densely packed sheet (FIG. 1). These sheets were incubated with 10×PBS for 30 minutes at 37° C. Each sheet was approximately 4 cm×4 cm and 100 microns thick.

Next, 1-Ethyl-3-(3-dimethylaminopropyl)-carbodiimide (EDC) and N-hydroxysuccinimide (NHS) (Life Technologies) were used to cross-link heparin molecules to aligned collagen sheets. Sodium salt of heparin (extracted from porcine intestinal mucosa, Celsus, Inc.) was dissolved in EDC-NHS solution (EDC to NHS ratio of 1.67 and pH 7-8 in deionized water) at heparin:collagen molar ratio of 80:1. Collagen sheets were soaked in heparin-crosslinking solution for 2 h. Subsequently, sheets were rinsed 3 times briefly and a fourth time for 15 min in deionized water. In a second cross-linking step, the procedure was repeated without heparin in an EDC-NHS 80% ethanol solution for 30 minutes to crosslink collagen molecules to each other to provide the sheet with mechanical robustness. Sheets were rinsed in the same manner and stored in PBS at 4 C.

To determine the amount of heparin crosslinked to collagen sheets, heparinized sheets were dissolved in 1 N HCl at 55° C. Dimethylmethylene Blue (DMMB) assay for sulfated glycosaminoglycans was used to measure the concentration of heparin in solution, which indicates the quantity of heparin that was crosslinked to collagen sheets. Otherwise, heparinized sheets were cut into discs using an 8 mm biopsy punch and discs were dried for 2 hours at 37° C. Each disc was incubated overnight in 150 ul of 2 mg/ml gentamicin sulfate solution (Sigma-Aldrich) at 4° C. to allow for electrostatic association of gentamicin to heparin. Additional collagen sheets were prepared in the exact same manner without heparin to serve as controls.

### Example II

Heparinization Confirmation

Heparinization of the collagen sheet was qualitatively and quantitatively analyzed using dimethyl methylene blue (DMMB) staining. Direct visualization of collagen film staining was used to qualitatively confirm successful heparinization of the films. To quantitatively measure the amount of heparin that was covalently crosslinked to the collagen films, heparinized films were dissolved in 1 N HCl at 85° C. and DMMB assay was used to measure the concentration of heparin in the solution.

### Example III

Antibacterial Efficacy of Collagen-Heparin-Gentamicin Sheets

Bacterial diffusion assays were performed using 4 mm-discs of sheets plated onto agar with 1×10⁸ Pseudomonas aeruginosa per plate. The test groups were sheets produced in accordance with Example I, and a control group used collagen sheets that had undergone gentamicin incubation but no heparinization. Radii of zones of inhibition were measured over 5 days.

### Example IV

Quantitation Of Gentamicin Loading

This example will show quantitative data of the kinetics of gentamicin loading onto heparin-collagen sheets.

### Example V

Delineation of Long-Term Gentamicin Release Profiles

This example will show data for gentamicin release profiles over a clinically-relevant time frame that are delinated using high performance liquid chromatography (HPLC).

### Example VI

Optimization of Heparin and Gentamicin Concentrations

This example will present data demonstrating the optimization of heparin and gentamicin loading concentrations with quantitative demonstration.

### Example VII

Bacterial Diffusion Assays

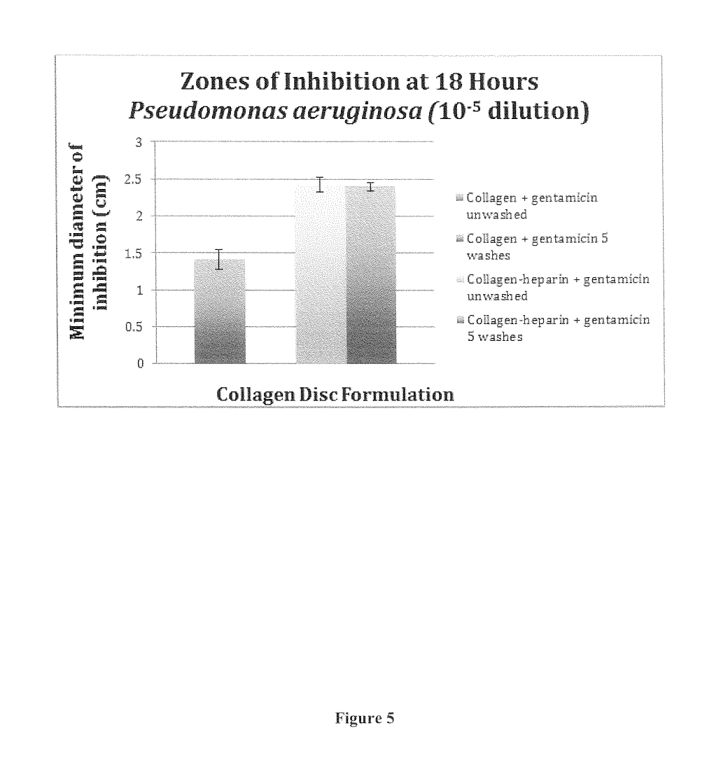

Following overnight incubation in gentamicin solution, test collagen discs and control collagen discs were each then divided into an “unwashed” group and a “5 washes” group. Washing antibiotic loaded discs as such served to test the affinity of the gentamicin to heparinized collagen. Discs in the 5 washes group were each rinsed in 1 mL of DI water 5 times. Next, Pseudomonas aeruginosa at 10-5\hat{ }-5 dilution was spread onto agar plates (100 ul per plate). Plates were dried for 60 minutes. Collagen discs were then blotted briefly using Kim Wipes prior to being placed onto the agar plates. Plates were stored at 37° C. and zones of inhibition were measured in diameter using digital calipers at 18 hours after plating and 5 days after plating. This assay was then repeated using moxifloxacin hydrochloride 0.5% (Vigamox solution) instead of gentamicin sulfate to assess the broader efficacy of heparinized collagen. See, FIG. 5.

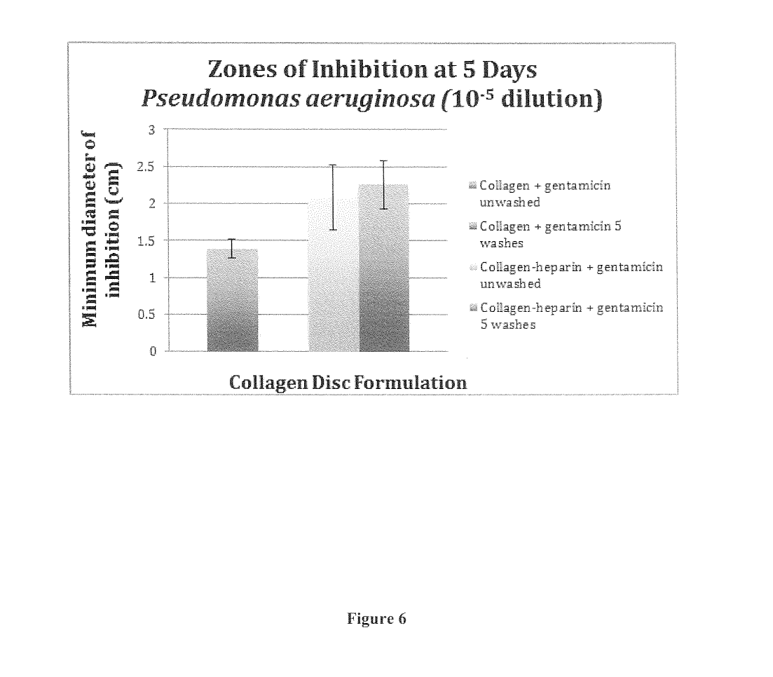

At 18 hours after plating, collagen-heparin sheets that had been washed demonstrated zones of inhibition against Pseudomonas aeruginosa of equal size to unwashed collagen-heparin sheets (mean diameter of 2.40 cm in the washed group vs. 2.42 cm in the unwashed group). Unwashed control collagen sheets also produced zones of inhibition, but of significantly smaller size than either group of collagen-heparin sheets (mean diameter of 1.42 cm). Washed control collagen sheets demonstrated no zones of inhibition. At 5 days after plating, some zones in the collagen-heparin groups had shrunken slightly, but the relative sizes of zones were sustained. See, FIG. 6.

### Example VIII

Moxifloxacin Diffusion Assay

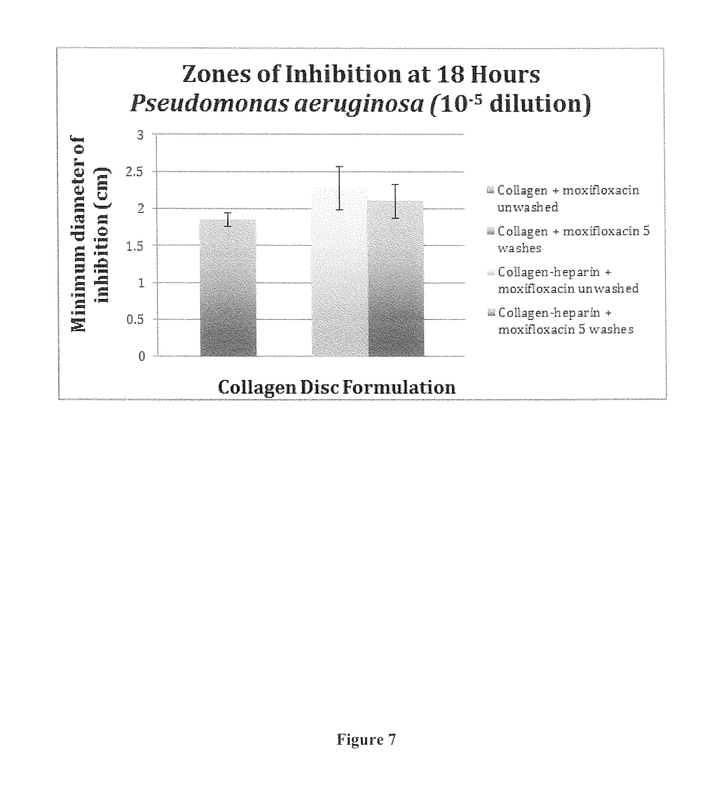

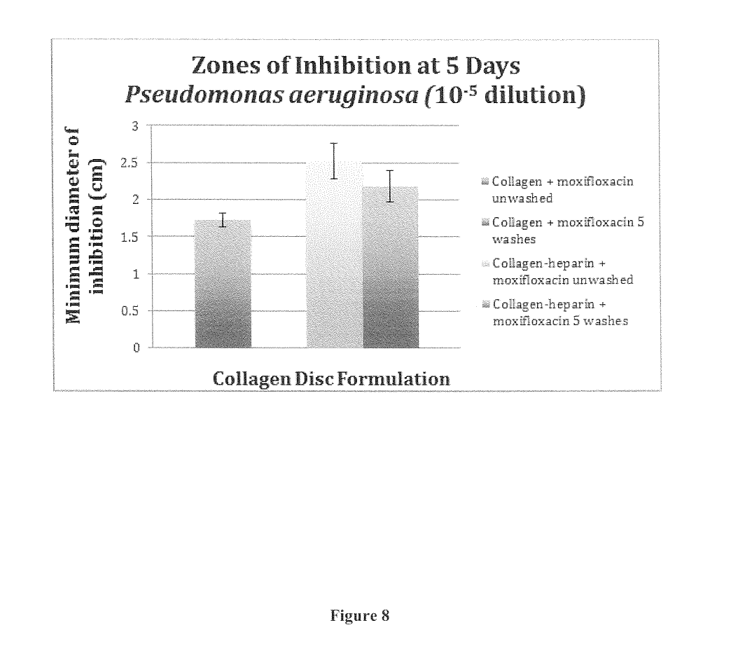

With moxifloxacin as well, at 18 hours after plating, collagen-heparin sheets that had been washed demonstrated zones of inhibition against Pseudomonas aeruginosa of similar size to unwashed collagen-heparin sheets (mean diameter of 2.10 for washed vs. 2.28 for unwashed). Unwashed control collagen sheets also produced zones of inhibition (mean diameter 1.85 cm). Washed control collagen sheets demonstrated no zones of inhibition. At 5 days after plating, zones were sustained for all groups with only minor change s in diameter. FIGS. 7 and 8.

### Example IX

Fabrication of Aligned Collagen Sutures

Electrochemically aligned collagen threads were fabricated as described herein. Briefly, two parallel electrode wires were circumferentially wound around a rotating disc. Dialyzed collagen solution (Collagen Solutions Inc., bovine dermis, telocollagen, 3 mg/ml) was applied on the top of the rotating disc in between the two electrodes. Electrical current was applied at 10 A to the electrodes which resulted in the generation of a pH gradient between the electrodes. Collagen molecules in different pH solutions acquire different charges (negative near the cathode and vice versa for the anode) and the electrostatic repulsion of molecules by the electrodes push the collagen molecules toward the isoelectric point. Liquid collagen transforms into compacted threads in less than a minute following which the thread is collected onto a rotating spool. The collagen thread diameter was 0.11±0.03 mm. The biophysical principles of collagen electrokinetics under pH gradients were modeled and discussed in detail in a prior study. J. A. Uquillas, O. Akkus, Modeling the electromobility of type-I collagen molecules in the electrochemical fabrication of dense and aligned tissue constructs, Ann. Biomed. Eng. 40 (2012) 1641-1653.

### Example X

Genipin Crosslinking

Genipin crosslinking enhances the strength of collagen threads to the level of the native tendon. Prior to genipin crosslinking, collagen suture was incubated in 1 PBS (Fisher scientific) for 6 h, and after that kept in isopropanol bath overnight. Following these steps aligned collagen sutures were crosslinked in genipin solution (0.625 g in 100 ml of 90% vol ethanol solution) for 72 h. Crosslinked aligned collagen sutures were washed thoroughly 3 times with deionized water, dried and kept at 4° C.

### Example XI

Peracetic Acid/Ethanol Treatment (Pet)

Genipin crosslinking limits the swelling ratio of collagen threads and usurps sites available for conjugation of heparin. This limitation was addressed by Pet process. Collagen sutures were incubated in peracetic acid solution for 4 h. Peracetic acid/ethanol solution consisted of 2% vol Peracetic acid (Sigma-Aldrich), 200 proof ethanol (Fisher Scientific) and deionized water (volume ratio 2/1/1). S. U. Scheffler, J. Scherler, A. Pruss, R. von Versen, A. Weiler, Biomechanical comparison of human bone-patellar tendon-bone grafts after sterilization with peracetic acid ethanol, Cell Tissue Bank. 6 (2005) 109-115. After 4 h the solution was removed and samples were rinsed thoroughly with deionized water 3 times for 15 min each time. Samples were dried and stored at 4° C.

### Example XI

Heparin Conjugation to ELAC Sutures

1-Ethyl-3-(3-dimethylaminopropyl)-carbodiimide (EDC) and N-hydroxysuccinimide (NHS) (Life Technologies) were used to crosslink heparin molecules to aligned collagen sutures. Sodium salt of heparin (extracted from porcine intestinal mucosa, Celsus, Inc.) was dissolved in EDC-NHS solution (EDC to NHS ratio of 0.72 and pH 7-8) at 3 different concentrations of 1 mg/ml (low), 3 mg/ml (medium) and 10 mg/ml (high). Collagen sutures were soaked (10 cm of thread in each 2 ml of solution) in heparin-crosslinking solution for 2 h. After that, collagen sutures were rinsed 3 times with deionized water for 15 min each time, dried completely and stored at 4° C. To find the amount of heparin which is crosslinked to collagen sutures, heparinized sutures were dissolved in 1 N HCl at 37° C. for 72 h. Dimethylmethylene Blue (DMMB) assay was used to measure the concentration of heparin in the solution which indicates the amount of heparin that is crosslinked to collagen sutures. Younesi et al., Acta Biomaterialia 41 (2016) 100-109.

### Example XIII

Mechanical Properties of ELAC Sutures

Sutures were tested in tension after genipin crosslinking, after Pet process and after heparinization (N=8/group, sample length=25 mm). Also, a group of sutures which were genipin crosslinked, peracetic acid treated, and/or crosslinked in EDC-NHS solution without heparin was tested to assess whether heparin contributes to mechanics of threads. Load-displacement curves were recorded using an ARES rheometer (TA instruments, New Castle, Del.). Briefly, samples were soaked in PBS for 24 h prior to testing. Samples were fixed onto the rheometer fixture with 10 mm gauge length and subjected to uniaxial tensile loading until failure at a strain rate of 10 mm/min. The load-displacement data were used to calculate the ultimate tensile strength (UTS) and Young's modulus of collagen sutures. Mechanical properties of ELAC sutures were also measured after 3 weeks of cell culture (N=8/group).

### Example IVX

Mechanical Properties of Lacerated Tendon Sutured with ELAC In Vitro

As the standard of care for flexor tendon repair, a nonabsorbable braided or monofilament suture is usually used as the core suture. A secondary epitendinous suture is applied close to the laceration site. J. H. Clare Langley, Focus on flexor tendon repair, J. Bone Joints Surg. (2009) 1-3.

In this study, ELAC was applied as the epitendinous suture to cadaveric tendons to assess whether it contributes to mechanical robustness of the baseline repair. The second digit flexor profundus tendon of chicken claw was cut with a scalpel. Two ends of the tendon were sutured together using a number 6-0 nylon core suture for load bearing purpose.

Three groups were included. In the first group, only the nylon core suture was applied. In the second group, ELAC suture was used as epitendinous suture in addition to the nylon core suture. In the third group, a nylon suture was used as the epitendinous suture in addition to the nylon core suture. Sutured tendon samples were tested in tension (N=3/group). Load-displacement curves were recorded using a materials test machine (Test Resources 800LE3-2, Test Resources Inc., MN, USA). Briefly, samples were kept hydrated via wet gauze with 1 PBS prior to testing for 24 h. Samples were fixed onto the fixture and subjected to uniaxial tensile loading until failure at a strain rate of 10 mm/min. The load-displacement data were collected and the load at failure is reported as a measure of the attachment strength attained by different suture types.

### Example XV

Swelling Ratio

Ten cm length of aligned collagen sutures from both untreated and peracetic treated group were weighed in the dry state. Samples were soaked in PBS solution for 24 h to attain the equilibrium swelling state. Weights of samples were measured again and the following formula was used to calculate the swelling ratio of treated collagen suture.

### Example XVI

PDGF-BB Release from Heparinized Collagen Sutures

PDGF-BB release from collagen sutures was measured with and without heparin. Samples were prepared 3 cm in length and fixed horizontally on a jig and suspended in air. An aliquot of PGDF-BB growth factor solution (containing 100 ng of the growth factor) was applied on samples with a micropipette. The solution was soaked and taken up completely by the originally dry sutures. Samples loaded as such were left to dry. Each sample was then placed in a low protein bind centrifuge tube and 2 ml of release media (0.1% bovine serum albumin and 0.1% NaN3 in PBS) was added in each tube and incubated at 37° C.

At predetermined time points (2, 4, 6 h, 1, 2, 3, 4, 5, 10, 15 days), the supernatant was removed and fresh release media was added. The cumulative amount of PDGF-BB in the release media from each sample was measured using a human PDGF-BB ELISA development kit (Peprotech, Rocky Hill, N.J., USA) based on calibration curves derived using known concentrations of the growth factors (N=3 for each group per time points). The total PDGF-BB was determined to be the sum of the PDGF-BB in the release media and the remaining growth factor in the heparinized collagen sutures as determined by ELISA assay.

### Example XVII

Isolation of Tendon-Derived Cells from Chicken Flexors

Chicken tendon tissue was dissected immediately post-mortem under a protocol approved by the Institutional Animal Care and Use Committee (IACUC) at Case Western Reserve University (protocol No. 2013-0075). Chicken is a well-established model for studying flexor-tendon because it has a similar tendon anatomy to human hands and the prominences of flexor tendons facilitate the surgery. Comiter et al., “High rate of vaginal extrusion of silicone-coated polyester sling,” Urology, vol. 63, no. 6, pp. 1066-1070, 2004. Cells were isolated from the second and the third digital flexor tendons of chickens (White leghorn chicken, aged 12 weeks and weighing approximately about 1-1.5 kg) following published protocols. Cao et al., “Bridging tendon defects using autologous tenocyte engineered tendon in a hen model” Plast. Reconstr. Surg. 110 (2002) 1280-1289. Three cm of the tendon was dissected and minced in small pieces using a scalpel in a petri dish. Tendon pieces were washed thoroughly with 1 PBS and then digested in 0.25% collagenase II (Worthington. Freehold, N.J.) in serum free Dulbecco's modified eagle medium (Gibco, Grand Island, N.Y.) for 12 h at 37° C.

The tissue fragments were separated by low-speed centrifugation at 100 RPM for 1 min and the supernatant with cells was seeded on T-75 flasks for cells to adhere for 48 h. Cultures were rinsed with media, cells were trypsinized and seeded at a density of 350,000 in T-75 flask in growth media (low glucose Dulbecco's modified eagle's medium, 10% Fetal bovine.

Epitendinous ELAC suture contributed to mechanical stabilization significantly beyond that is provided by the core suture. (a) A comparison between 6-0 standard nylon suture and aligned collagen suture. (b) A lacerated flexor tendon of chicken is sutured with aligned collagen suture to demonstrate the feasibility of suturing. (c) Ultimate failure load of lacerated tendon with epitendinous collagen sutures is greater than that of the tendon repaired with only core nylon suture, and, significantly less than the tendon repaired by the epitendinous 6-0 nylon sutures. (d) Typical load-displacement curves of lacerated tendons sutured under different conditions (Scale bar: 10 mm). Younesi et al., Acta Biomaterialia 41 (2016) 100-109.

### Example XVIII

Effect of Heparinization on Cell Proliferation on ELAC Sutures

Cells were seeded on 12 mm length suture samples as follows: 1) suture samples without heparinization, 2) low-level of heparin conjugation (1 mg/ml), and 3) high-level of heparin conjugation (10 mg/ml). Samples were sterilized in 70% v/v ethanol solution overnight and washed with PBS 3 times each 15 min. Ten pieces of 12 mm samples were placed in low attachment 24 well-plates.

Tenocytes were seeded at a density of 20,000 per cm² on samples in growth media (low glucose Dulbecco's modified eagles medium, 10% fetal bovine serum, 1% penicillin/streptomycin, 1% L-glutamine, 50 mg ascorbic acid). After 4 h, nonattached cells were collected by changing the media and counted to calculate the number of attached cells.

After 7 days, Alexa fluor 488 (Life Technologies) for actin staining and DAPI (40,6-diamidino-2-phenylindole) staining for cells nuclei were performed on samples. Cell numbers on samples were counted to calculate the proliferation of the cells on heparinized aligned collagen sutures. (N=3 per group).

### Example IXX

Effect of PDGF-BB Release from Heparinized ELAC Sutures on Cell Proliferation

Three groups were included: ELAC sutures samples, heparinized ELAC sutures samples with PDGF-BB (samples treated in 10 mg/ml heparin and the 100 ng PDGF-BB was loaded on each 3 cm of suture samples), and strips of uncompacted collagen gel were prepared as a reference point to determine the effect of electrocompaction and alignment.

Ten pieces of samples/group, each 30 mm in length, were soaked in serum supplemented media for 30 min and placed in 6 well-plates as 10 samples per well. Cells were seeded as described earlier. After 4 h, non-attached cells were collected by changing the media and counted to calculate the number of attached cells to samples. Alamar blue assay (Life Technologies) was performed on samples at day 7 to investigate the effect of PDGF-BB on metabolic activities of tendon-derived cells. Also, 3 samples per group were lysed and DNA quantification was performed to measure the cell proliferation at day 7 (N=3 per group, averages and standard deviations were reported).

### Example XX

Collagen Solubilized to the Media

Tendon derived cells were seeded on uncompacted collagen gel samples, ELAC sutures samples and ELAC sutures samples with PDGF-BB. Samples were cultured in ascorbic acid free media for 21 days. Every 3 days the media was collected and fresh media was replaced.

All collected media for each group from different time points were mixed and total synthesized collagen by cells over 21 days was measured using the Sircol assay kit for measuring the amount of collagen in the culture medium (Sircol Soluble Collagen Assay, Biocolor Life Science Assays). A micro-plate reader (Spectramax M2, Molecular Devices) was used to measure the absorbance of the samples at 555 nm wavelength. Total collagen amount synthesized by cells were calculated by using the calibration curve plotted based on standard samples absorbance. Tests were repeated in triplets.

### Example XXI

Histology

Tendon-derived cells were seeded on uncompacted collagen gel samples, ELAC sutures samples and ELAC sutures samples with PDGF-BB as described. Samples were cultured in the growth media supplemented with ascorbic acid for 21 days. Samples were fixed in 10% formalin solution at room temperature for histological processing. Fixed samples were placed through a series of increasing ethanol solutions and xylene steps to clear the constructs. Samples were then embedded in paraffin and cut into 5 lm sections. General histological staining of hematoxylin and eosin (H&E) and Masson-trichrome were performed on xylene-cleared sections to highlight cell morphology and collagen production, respectively.

### Example XXII

Effect of PDGF-BB Release from Heparinized ELAC Sutures on Tenogenic Gene Expression

The same three groups and seeding method were followed as described in the proliferation studies. At days 7, 14 and 21, the cells on samples were lysed using TRIzol Reagent (Life Technologies, NY, USA) and total RNA were extracted according to manufacturer's instructions. RNA was reverse-transcribed to cDNA (Applied Biosystems, CA, and USA). Taqman gene expression assays for scleraxis, type-I collagen, tenomodulin and COMP were used with the cDNA to evaluate the expression of genes using real-time PCR (Applied Biosystems 7500 Real-Time PCR System). The relative fold-change in target gene expression was quantified using the 2\hat{ }(−ddCt) method (TaqMan Gene Expression Assays Protocol, Applied Biosystem, life technologies) by normalizing the target gene expression to RPLP-0 and relative to the expression on the random collagen at each time points. PCR experiments were run in triplicate at separate times (N=3).

### Example XXIII

Statistical Analysis

Mechanical test data were analyzed with one-way ANOVA with Tukey's pairwise comparison to determine significant differences between genipin crosslinked sutures, EDS/NHS crosslinked sutures, peracetic treated sutures and heparinized sutures and also for significant differences in the failure loads between tendons sutured with only nylon core suture, core suture plus epitendinous ELAC suture and core nylon suture plus epitendinous nylon suture. Same analysis was used for cell proliferation, collagen production and gene expression data to find the significant differences in proliferation of cells on genipin crosslinked sutures, sutures with low (1 mg/ml) and high (10 mg/ml) concentrations of heparin. Data are reported as mean±st. dev. Significance is reported at the level of p<0.05. 3.

49. A medical device encased by a heparin crosslinked collagen sheet coating.

50. The medical device of item 49, wherein said coating further comprises gentamicin attached to said heparin.

51. The medical device of item 49, wherein said gentamicin-heparin attachment is an electrostatic association.

52. The medical device of item 49, wherein said coating further comprises a drug or compound.

53. The medical device of item 52, wherein said drug or compound is an antibiotic.

54. The medical device of item 53, wherein said antibiotic is selected from the group consisting of aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof. In one embodiment, the medical device comprises a head implant. In one embodiment, the medical device comprises a neck implant. In one embodiment, the medical device is a titanium mandibular plate.

55. A method, comprising:
a) providing;
   i) a patient exhibiting a medical condition and a plurality of bacteria;
   ii) a medical device encased by a heparin crosslinked collagen sheet coating, said device being configured to treat said medical condition; and
b) contacting said patient with said medical device under conditions such that said plurality of bacteria are subjected to a bacteriostatic effect.

56. The method of item 55, wherein said contacting further comprises that said plurality of bacteria are subjected to a bacteriocidal effect.

57. The method of item 55, wherein said coating further comprises gentamicin attached to said heparin.

58. The method of item 57, wherein said gentamicin-heparin attachment is an electrostatic association.

59. The method of item 55, wherein said coating further comprises a drug or compound.

60. The method of item 59, wherein said drug or compound is an antibiotic.

61. The method of item 60, wherein said antibiotic is selected from the group consisting of aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof. In one embodiment, the medical device comprises a head implant. In one embodiment, the medical device comprises a neck implant. In one embodiment, the medical device is a titanium mandibular plate.

What is claimed is:

1. An electrochemically aligned and compacted (ELAC) collagen suture comprising at least one growth factor and heparin.

2. The ELAC suture of claim 1, further comprising a crosslinker.

3. The ELAC suture of claim 2, wherein said crosslinker is genipin.

4. The ELAC suture of claim 1, wherein said at least one growth factor is conjugated to said ELAC suture, or wherein said at least one growth factor saturates said ELAC suture, or wherein said heparin is attached to said at least one growth factor, or wherein said heparin attachment to said at least one growth factor is an affinity bonding attachment, or wherein said at least one growth factor is platelet derived growth factor, or wherein said at least one growth factor is selected from the group consisting of basic fibroblast growth factor, transforming growth factor-β1, vascular endothelial growth factor and insulin-like growth factor 1, or wherein said ELAC suture further comprises a plurality of aligned threads, or wherein said ELAC suture is a load-bearing ELAC suture, or wherein said ELAC suture further comprises a heparinized collagen matrix, or wherein said ELAC suture further comprises a plurality of genipin crosslinks, or wherein said heparin is attached to said ELAC suture with peracetic acid in combination with 3-dimethylaminopropyl)-3-ethylcarbodiimide-N-hydroxysuccinimide.

5.-10. (canceled)

11. The ELAC suture of claim 4, wherein said conjugation of said at least one growth factor to said ELAC suture is reversible.

12. The ELAC suture of claim 1, wherein said heparin is attached to said at least one growth factor, wherein said attachment of said at least one growth factor to the heparin is reversible.

13.-16. (canceled)

17. A method, comprising:
a) providing;
i) a patient exhibiting a wound, said wound comprising biological cells; and ii) an electrochemically aligned and compacted (ELAC) collagen suture comprising a plurality of at least one growth factor and heparin;
b) suturing said wound with said suture to create a sutured wound; and
c) healing said sutured wound faster than a conventional suture.

18. The method of claim 17, wherein said ELAC suture further comprises a crosslinker.

19. The method of claim 18, wherein said crosslinker is genipin.

20. The method of claim 17, wherein said wound is a lacerated tendon, or wherein said conventional suture is a nylon suture, wherein said conventional suture is a silk suture, or wherein said method further comprising releasing said plurality of at least one growth factor from said ELAC suture, or wherein said at least one growth factor release induces proliferation of said biological cells, or wherein said ELAC suture induces collagen production by said biological cells, or wherein said ELAC suture is a load-bearing suture, or wherein said ELAC suture upregulates Collagen I, SCX, COMP, and TNMD gene expression in said biological cells, or wherein said biological cells are selected from the group consisting of tenocytes, tenoblasts and mesenchymal stem cells, or wherein said at least one growth factor is conjugated to said ELAC suture, or wherein said at least one growth factor saturates said ELAC suture, or wherein said heparin is attached to said at least one growth factor, or wherein said at least one growth factor is platelet derived growth factor, or wherein said at least one growth factor is selected from the group consisting of basic fibroblast growth factor, transforming growth factor-β1, 5 vascular endothelial growth factor and insulin-like growth factor 1, or wherein said ELAC suture further comprises a plurality of aligned threads, or wherein said ELAC suture is a load-bearing ELAC suture, or wherein said ELAC suture further comprises a heparinized collagen matrix, wherein said ELAC suture further comprises a plurality of genipin crosslinks.

21.-23. (canceled)

24. The method of claim 17, said method further comprising releasing said plurality of at least one growth factor from said ELAC suture, and wherein said at least one growth factor release is for approximately fifteen (15) days.

25.-32. (canceled)

33. The method of claim 17, wherein said heparin is attached to said at least one growth factor, and wherein said heparin attachment to said at least one growth factor is an affinity bonding attachment.

34.-36. (canceled)

37. The method of claim 17, wherein said at least one growth factor is conjugated to said ELAC suture, wherein said conjugation of said at least one growth factor to said ELAC suture is reversible.

38. The method of claim 17, wherein said heparin is attached to said at least one growth factor, wherein said attachment of said at least one growth factor to said heparin is reversible, or wherein said heparin is attached to the ELAC suture with peracetic acid in combination with 3-dimethylaminopropyl)-3-ethylcarbodiimide-N-hydroxysuccinimide.

39.-42. (canceled)

43. A biologic antibacterial coating composition comprising a heparin crosslinked collagen sheet.

44. The coating of claim 43, wherein said collagen sheet further comprises gentamicin attached to said heparin.

45. The coating of claim 44, wherein said gentamicin-heparin attachment is an electrostatic association.

46. The coating of claim 43, wherein said collagen sheet further comprises a drug or compound.

47. The coating of claim 46, wherein said drug or compound is an antibiotic.

48. The coating of claim 47, wherein said antibiotic is selected from the group consisting of aminoglycosides, carbapenems, ceftazidimes, cefepimes, ceftobiproles, fluoroquinolones, piperacillins, tazobactams, ticarcillins, clavulanic acids, erythromycins, clindamycins, gentamycins, tetracyclines, meclocyclines, sulfacetamides, penicillins, vancomycins, chlortetracyclines, metronidazoles, tinidazoles, cephamandoles, latamoxefs, cefoperazones, cefmenoximes, furazolidones, chloramphenicols, aminoglycosides, cephalosporins, rifamycins, lipiarmycins, quinolones, sulfonamides, macrolides, lincosamides, cyclic lipopeptides (such as daptomycin), glycylcyclines (such as tigecycline), oxazolidinones (such as linezolid), and lipiarmycins (such as fidaxomicin), and derivatives thereof.

49.-61. (canceled)
